(19) United States
(12) Patent Application Publication
(10) Pub. No.: US 2024/0173561 A1
(43) Pub. Date: May 30, 2024

(54) MICROWAVE TREATMENT OF TISSUE

(71) Applicant: Emblation Limited, Alloa (GB)

(72) Inventors: Gary Beale, Alloa (GB); Eamon McErlean, Alloa (GB); Shailesh Joshi, Alloa (GB); Matthew Kidd, Alloa (GB)

(21) Appl. No.: 18/562,654

(22) PCT Filed: May 20, 2022
(86) PCT No.: PCT/GB2022/051280

§ 371 Date: Nov. 20, 2023

Related U.S. Application Data
(60) Provisional application No. 63/191,463, filed on May 21, 2021.

(51) Int. Cl. A61N 5/02; C12N 13/00

(52) U.S. Cl. CPC A61N 5/025; C12N 13/00

(57) ABSTRACT

The disclosure is based on the finding that microwave energy may be used to modulate, for example up- or down-regulate the expression of certain genes. For example, where a disease or condition is associated with the aberrant expression of a particular gene or genes, microwave energy may be used to modulate the expression of those genes, thereby resolving and/or improving one or more of the symptoms of the disease or condition. Disclosed is a microwave system or microwave-generating apparatus, for use in a method of modulating the expression of one or more genes. Also disclosed are uses of microwave energy for modulating the expression of one or more genes in tissues, tissue samples and biopsies.

### FIELD

The present disclosure provides microwave generating apparatus, systems and methods for modifying gene expression and for treating diseased tissues. The disclosed methods rectify tissue dysregulation, reset dysregulated intrinsic and extrinsic pathways and restore tissue homeostasis by restoring gene expression of the dysregulated tissue such as epithelial tissue. The disclosed methods may also promote tissue repair, healing and regeneration.

### BACKGROUND

A system and method for treating a diseased tissue using an electromagnetic energy system, including microwave energy is hereby described. The said system and method ameliorate tissue inflammation, rectify tissue dysregulation, reset dysregulated intrinsic and extrinsic pathways and restore tissue homeostasis to promote tissue repair, tissue healing and tissue regeneration by restoring aberrantly upregulated or downregulated genes in the inflamed tissue to a normal baseline level such as of healthy tissue.

Inflammation is widely regarded as a critical component in the carcinogenesis and tumor progression of many cancer types. Prolonged inflammation often leads to carcinomas which are malignant neoplasms originating from the dysregulated epithelial tissue and account for almost 90% of all cancer types such as squamous cell carcinoma, transitional cell carcinoma, renal cell carcinoma and adenocarcinomas. For example, persistent inflammation of intestinal mucosa in the IBD (inflammatory bowel disease) such as ulcerative colitis (UC) often leads to metastatic colorectal cancers. Other examples include uncontrolled inflammation of ovarian epithelial leading to ovarian cancer or chronic pancreatitis leading to pancreatic cancer or persistent inflammation of the urothelial epithelium lining developing into squamous cell carcinoma of the bladder [1] [2] [3] [4] [5].

Inflammation is known to sustain the proliferation and survival of malignant transformed cells and is able to promote angiogenesis and metastatic processes. The link between inflammation and cancer depends on intrinsic and extrinsic pathways. Most often in the chronic stage of the inflammation key biomarkers participating in important cancer pathways such as PI3K (Phosphatidylinositol 3′-kinase (PI3K)-Akt), MAPK (Mitogen-activated protein kinase), Notch, TGF-B (Transforming growth factor-beta), HedgeHog, JAK/STAT (Janus kinase/signal transducers and activators of transcription) are dysregulated i.e. either aberrantly upregulated or downregulated. Dysregulated pathways contribute to promote tumor growth, progression, and metastatic spread [3] [6].

Common methods of treating inflammatory conditions include anti-inflammatory agents comprising steroids, enzymes, biological drugs, aminosalicylates, antibiotics and immunomodulators which are usually directed towards reducing inflammation of the tissue but fail to address the long-term remission of the diseases. However, persistent inflammation is capable of altering the efficacy of therapeutic agents and these conservative therapies are often accompanied with major side effects. Ultimately their failure leads to invasive surgery which can lead to more debilitating complications.

Inflammatory conditions are typically characterized by not just inflammatory cell accumulation but more importantly by severe damage of the epithelial layer suggesting epithelium healing is the most significant prognostic factor in long term remission of such diseases. In an otherwise healthy person, epithelial cells are renewed every 2-3 days by shedding of old cells and generation of new cells. This balance is disrupted during injury causing more shedding than regeneration leading to more epithelial gaps and barrier dysfunction. Repeated damage and injury to the epithelium leads to chronic inflammation and eventually to a metastatic carcinoma. In such inflammatory conditions, epithelial tissues display increased inflammatory activity often measured with high levels of inflammatory cytokines, cytokine storm, oxidative stress, lymphocyte count, transcription factors etc. [7] [8] [9] [13].

The normal epithelium restoration process is guided by three mechanisms of healing viz. 1) epithelial restitution, 2) epithelial cell proliferation and 3) epithelial cell differentiation. In the acute phase of the injury adjacent healthy epithelial cells, for example intestinal epithelial cells (IECs) in the mucosal epithelium, migrate to cover the injured area to re-establish the integrity of the epithelial layer and to reconstitute the barrier between the intestinal lumen and the submucosa, a process known as epithelial restitution. In the later stages of healing, epithelial cell proliferation takes over to replenish decreased cell count followed by the third phase of maturation and differentiation of epithelial cells. These three phases may also overlap. In case of deeper lesions or penetrating injuries additional repair mechanisms involving inflammatory processes and non-epithelial cells support the healing process [11].

Epithelial inflammation and its healing, repair and regeneration is regulated by broad spectrum of regulatory factors such as growth factors, cytokines, proteins, regulatory peptides, peptide growth factors, interleukins, interferons etc. These modulatory factors play an essential role in a complex cascade in regulating epithelial cell functions and preserve normal homeostasis and integrity of the epithelia. In a diseased state, the genes encoding these regulatory factors are dysregulated i.e. aberrantly upregulated or downregulated thus dysregulating normal functionality of the epithelia. Therefore, restoring abnormal gene counts of such multiple regulatory factors to a normal baseline level such as that of the healthy tissue is a method of treatment. Furthermore, maintaining a crosstalk between inflammatory signals and regenerative signals in tissues such as epithelial tissue is a key factor in effectively treating inflammatory conditions.

Most current treatments and therapies in treating inflammatory conditions are directed towards reducing inflammation by typically targeting a one or more than one biomarker and have shown limited success in long term remissions of diseases. A series of study reviews have shown complete healing, repair and regeneration of the epithelial layer is a common prognostic factor for long-term remission of inflammatory conditions at both endoscopic and microscopic level [7] [8] [16].

Thus, an effective approach to treating inflammatory conditions and dysregulation of the epithelium by focusing on rectifying tissue dysregulation, resetting dysregulated intrinsic and extrinsic pathways and restoring normal tissue homeostasis to promote epithelial repair, leading to the healing and regeneration of the tissue is needed to treat patients more effectively. The present invention provides such an approach by using an energy-based treatment therapy to promote tissue repair, and healing in particular, epithelial tissue repair, healing and regeneration through immunomodulatory and therapeutic effects at a genomic level by rectifying tissue dysregulation and resetting dysregulated intrinsic and extrinsic pathways to restore normal tissue homeostasis. The present invention provides a system and method for restoring aberrantly dysregulated levels of gene biomarkers in the diseased tissue to a normal level, where a normal level corresponds to the gene count in healthy tissue, absent of disease.

Methods for stimulating epithelial cell proliferation and regeneration in treating inflammatory conditions by administering a pharmacological compound alone or in combination such as gastrointestinal proliferative factor (GIPF) (U.S. Pat. No. 7,951,381B2) [17], purple non-sulfur bacteria (U.S. Pat. No. 9,737,573B2) [18], TGF-p 3 (AU2006268091C1) [19], Anti-MET antibody (US20190315873A1) [20], isolated polypeptides (U.S. Pat. No. 9,855,313B2) [21], HGF Hepatocyte Growth Factor (U.S. Pat. No. 5,972,887A) [22], 17ß-estradiol (WO2020/245277A1) andRspo1 agent (U.S. Pat. No. 9,827,290B2) are documented. Pharmacological agents have also shown benefits in modulating gastrointestinal epithelium proliferation through the Wnt signaling pathway (US 20050169995A1) [25]. A method of administering a modulating agent using gene editing system such as CRISPR to modulating the integrity of the intestinal epithelia by altering the expression of an intestinal gene such as Clorfl06 in treating inflammatory disease is known (WO2019018410A1) [26].

Inventions comprising energy-based devices and methods like electromagnetic systems for example microwave hyperthermia by selectively raising temperature of the tissue in other regenerative applications such as enhancing wound healing have been shown in the past (AU2007330615B2) and (U.S. Pat. No. 7,967,839B2) [28]. However, these inventions are based on causing tissue coagulation and destructive thermal damage and are limited to accelerate wound closure or wound sealing and fixing or fusing of tissues and implants.

Hezi-Yamit et al teaches energy-based methods to perform a destructive thermal ablation at 65° C. to increase IL-10 expression level at or near the target site to treat inflammatory conditions such as IBD (US 2015/0126978 A1) [29].

Further, energy based systems and methods such as using electrical energy and microwave energy to ablate tissues such intestinal tract at 60°−90° C. have been provided (US 2015/0141987 A1) [30], U.S. Ser. No. 10/349,998 B2 [31], (WO 2017/087191 A1) [32]. These methods which are claimed to offer potential therapeutic benefits to patients suffering from inflammatory conditions are promising, however the ablative and necrotic temperatures in the excess of 60° C., present risk and may be damaging with a substantial conductive thermal spread in the tissue depth and include side effects of scarring, which may possibly aggravate the disease.

The destructive nature of these treatments may also destroy significant amounts of healthy epithelial tissue surrounding the diseased lesions compromising the immune response that is essential in ameliorating chronic inflammations.

Moreover, these documented methods fail to restore tissue integrity and rectify tissue dysregulation which is essential in the long-term remission of inflammatory conditions which otherwise lead to tissue dysregulation and metastasis.

The present invention provides an energy-based system and method to treat and prevent inflammatory conditions, in particular inflammatory conditions related to the epithelial tissue. The system and methods presented herein provide immunomodulatory therapeutic effects to rectify tissue dysregulation and reset dysregulated intrinsic and extrinsic pathways to restore tissue homeostasis. The said system and methods promote tissue repair, healing and regeneration for example of epithelial tissue at a genomic level. The system and methods provided herein prevent development of chronic inflammation into carcinogenesis and metastatic cancers.

### SUMMARY

The present invention is based on the finding that microwave energy may be used to modulate, for example up- or down-regulate the expression of certain genes. For example, where a disease or condition is associated with the aberrant expression of a particular gene or genes, microwave energy may be used to modulate the expression of those genes, thereby resolving and/or improving one or more of the symptoms of the disease or condition.

In a first aspect, there is provided a microwave system or microwave-generating apparatus, for use in a method of modulating the expression of one or more genes.

The disclosure further provides microwave energy for use in a method of modulating the expression of one or more genes.

There is also provided a method of modulating the expression of one or more genes, said method comprising administering microwave energy to a subject in need thereof.

A microwave generator or system of this disclosure comprises a microwave generator; a controller configured to control the microwave generator to generate microwave energy having a selected operational frequency or range of frequencies; a microwave energy conduit cable configured to deliver the microwave energy to a microwave antenna extending from or coupled to a distal end of the microwave energy conduit cable; and a microwave antenna.

A microwave generator or system of this disclosure can be used to administer microwave energy to a diseased tissue, for example a diseased epithelial tissue. As described, this may not only lead to the modulated expression of one or more gene(s) but may also yield thermal and non-thermal effects within the tissue.

The subject may be any human or animal subject.

The subject may be suffering from (or susceptible/predisposed to) an inflammatory disease or condition.

The subject may harbour a diseased tissue exhibiting the symptoms of one or more diseases. The diseased tissue may exhibit symptoms characteristic of an inflammatory condition. The diseased tissue may comprise one or more dysregulated gene(s) and/or pathways. In such cases, a microwave-based method of this disclosure may be used to reset those dysregulated gene(s) and/or pathway(s).

The subject may harbor a damaged, wounded or injured tissue. A microwave-based method of this disclosure may promote tissue repair, healing and regeneration.

The subject may be suffering from (or susceptible/predisposed to) a disease or condition which is caused by and/or associated with, the aberrant expression of one or more of the genes listed in Table 1 below:

TABLE 1
genes, the expression of which can
be modulated by microwave energy

| | | Immunomodulatory pathway |
|---|---|---|
| Gene | Official full name | participation |
| IL8 | interleukin 8 | Chemokine Signaling |
| | | Cytokine Signaling |
| | | Host-pathogen Interaction |
| | | NF-kB Signaling |
| | | NLR signaling |
| | | TLR Signaling |
| SOCS3 | suppressor of cytokine | Adaptive Immune System |
| | signaling 3 | Cytokine Signaling |
| | | Host-pathogen Interaction |
| | | MHC Class I Antigen |
| | | Presentation |
| | | TNF Family Signaling |
| | | Type I Interferon Signaling |
| | | Type II Interferon Signaling |
| EGR1 | early growth response 1 | Cytokine Signaling |
| | | Host-pathogen Interaction |
| | | Lymphocyte Activation |
| | | Transcriptional Regulation |
| | | Type I Interferon Signaling |
| CD79A | Cluster of differentiation | Adaptive Immune System |
| | 79A | B cell Receptor Signaling |
| | | Lymphocyte Activation |
| IL1B | interleukin 1, beta | Cytokine Signaling |
| | | Host-pathogen Interaction |
| | | Innate Immune System |
| | | Lymphocyte Activation |
| | | NF-kB Signaling |
| | | NLR signaling |
| | | Oxidative Stress |
| | | Th17 Differentiation |
| | | TNF Family Signaling |
| | | TLR Signaling |
| TNFRSF13C | tumor necrosis factor | Host-pathogen Interaction |
| | receptor superfamily, | Cytokine Signaling |
| | member 13C | Lymphocyte Activation |
| | | NF-kB Signaling |

In view of the above, the disclosure provides:
a method of modulating the IL8 gene;
a method of modulating the SOCS3 gene;
a method of modulating the EGR1 gene;
a method of modulating the CD79A gene;
a method of modulating the IL1B gene;
a method of modulating the TNFRSF13C gene;
said method comprising exposing the gene to microwave energy. In one teaching the method may comprise exposing any of the abovementioned gene(s) gene to an amount or dose of microwave energy sufficient to modulate, for example, up- or down-regulate, the expression of that relevant gene. A method of this type may be applied to the modulation of gene expression in a tissue, in such cases, the method may comprise exposing a tissue exhibiting the aberrant expression of one or more gene(s), to microwave energy at a dose sufficient to modulate the expression of one or more of those gene(s) in the tissue. The tissue may be a diseased tissue. A method of this type may be used to restore the expression of any of the gene(s) described herein.

Moreover, the disclosure provides a method of treating or preventing a disease or condition caused by and/or associated with:
aberrant expression of the IL8 gene
aberrant expression of the SOCS3 gene
aberrant expression of the EGR1 gene
aberrant expression of the CD79A gene
aberrant expression of the IL1B gene
aberrant expression of the TNFRSF13C gene;
said method comprising administering a subject in need of treatment, with microwave energy. The microwave energy may be administered to the subject at a dose sufficient to modulate the expression (i.e. up- or down-regulate and/or restore the expression) of the relevant gene.

It should be understood that where a particular disease or condition is associated with the upregulation of a gene, microwave energy may be used to downregulate the expression of that gene.

Conversely, where a particular disease or condition is associated with the downregulation of a gene, microwave energy may be used to upregulate the expression of that gene.

Moreover, the microwave energy-based methods of this disclosure may be used to treat or prevent a particular disease or condition by restoring or normalising expression of aberrantly dysregulated gene(s), where the act of restoring or normalising involves modulating an aberrantly expressed or dysregulated gene towards a normal, healthy or baseline level of expression. It should be noted that a normal, healthy or baseline level of gene expression may be similar to the level of expression of the same gene as observed in a healthy tissue.

A subject to be administered a microwave-based treatment according to this disclosure, may be suffering from (or susceptible/predisposed to) a disease or condition which is caused by and/or associated with, the aberrant expression or functioning of one or more cellular pathway events. The inventors have discovered that microwave energy can be used to modulate these pathways. Without being bound by theory, it is suggested that microwave energy modulates the expression of one or more genes associated with these pathways and can therefore be used to restore or normalise the expression and/or function of any relevant pathway. * Terms restore and normalise may be interchanged in the document.

In view of the above, the term modulate means the up- or down-regulation of any given gene. The present invention is based on the finding that microwave energy can be used to modulate the expression of certain genes. For diseases characterised by the upregulation of some of these genes, microwave energy may be used to downregulate expression thereby treating the disease and/or a symptom thereof. Conversely, for diseases characterised by the downregulation of some of these genes, microwave energy may be used to upregulate expression thereby treating the disease and/or a symptom thereof.

Table 2 provides an indication of the specific effect of microwave energy on certain specific genes.

Table 2 below:

| Up-regulated | Down-regulated |
|---|---|
| KIT | IL8 |
| BAD | SOCS 3 |
| ID4 | EGR1 |
| RUNX1T1 | CD79A |
| AKT3 | IL1B |
| | TNFRSF13C |
| | GADD45B |
| | Notch3 |
| | CCND2 |
| | WNT5A |

In view of the above, the disclosure provides:
a method of upregulating the KIT gene;
a method of upregulating the BAD gene;
a method of upregulating the ID4 gene;
a method of upregulating the RUNX1T1 gene;
a method of upregulating the AKT3 gene;
said method comprising exposing the one or more of the abovementioned gene(s) to microwave energy. The gene may be exposed to microwave energy at an amount or at a dose described herein. In one teaching the method may comprise exposing the gene to an amount or dose of microwave energy sufficient to modulate, for example, upregulate, the expression of that gene. A method of this type may be applied to the upregulation of gene expression in a tissue, in such cases, the method may comprise exposing a tissue to microwave energy at a dose sufficient to upregulate the expression of the relevant gene in the tissue. The tissue may be a diseased tissue. A method of this type may be used to restore the expression of gene. The expression of a gene may be upregulated to the extent that the upregulated expression matches the expression of the same gene in a normal or healthy tissue.

Moreover, the disclosure provides a method of treating or preventing a disease or condition caused by and/or associated with:
downregulated expression of the KIT gene; and/or
downregulated expression of the BAD gene; and/or
downregulated expression of the ID4 gene; and/or
downregulated expression of the RUNX1T1 gene; and/or
downregulated expression of the AKT3 gene;
said method comprising administering a subject in need of treatment, microwave energy. The microwave energy may be administered to the subject in an amount of at a dose as described herein. The microwave energy may be administered at a dose sufficient to upregulate and/or restore the expression of the relevant gene. Note, the expression of a gene may be upregulated and/or restored to the extent that the upregulated/restored expression matches the expression of the same gene in a normal or healthy tissue.

The disclosure also provides:
a method of downregulating the IL8 gene;
a method of downregulating the SOCS3 gene;
a method of downregulating the EGR1 gene;
a method of downregulating the CD79A gene;
a method of downregulating the IL1B gene;
a method of downregulating the TNFRSF13C gene;
a method of downregulating the GADD45B gene;
a method of downregulating the Notch3 gene;
a method of downregulating the CCND2 gene;
a method of downregulating the WNT5A gene;
said method comprising exposing the gene to microwave energy. The gene may be exposed to microwave energy at an amount or at a dose described herein. In one teaching the method may comprise exposing the gene to an amount or dose of microwave energy sufficient to downregulate the expression of the relevant gene. A method of this type may be applied to the downregulation of gene expression in a tissue. In such cases, the method may comprise exposing a tissue to microwave energy at a dose sufficient to downregulate the expression of that gene in the tissue. The tissue may be a diseased tissue. A method of this type may be used to restore the expression of gene. The expression of a gene may be downregulated to the extent that the downregulated expression matches the expression of the same gene in a normal or healthy tissue.

Moreover, the disclosure provides a method of treating or preventing a disease or condition caused by and/or associated with:
upregulated expression of the IL8 gene;
upregulated expression of the SOCS3 gene;
upregulated expression of the EGR1 gene;
upregulated expression of the CD79A gene;
upregulated expression of the IL1B gene;
upregulated expression of the TNFRSF13C gene;
upregulated expression of the GADD45B gene;
upregulated expression of the Notch3 gene;
upregulated expression of the CCND2 gene;
upregulated expression of the WNT5A gene;
said method comprising administering a subject in need of treatment, microwave energy. The microwave energy may be administered to the subject at a dose sufficient to downregulate and/or restore the expression of the relevant gene. Note, the expression of a gene may be downregulated and/or restored to the extent that the downregulated/restored expression matches the expression of the same gene in a normal or healthy tissue.

Any modulation of the expression of a gene by microwave energy may be assessed relative to the expression of that gene either in normal healthy tissue and/or in diseased tissue. In a diseased tissue, the expression of certain genes may be higher than in normal healthy tissue. Microwave energy may be used to lower the expression of those genes, taking the level of expression down and towards the normal, baseline or healthy level. In other cases, a diseased tissue, may exhibit lower expression of certain genes as compared to the expression of the same genes in a healthy or normal tissue. Microwave energy may be used to raise the expression of those genes, taking the level of expression up and towards the normal, baseline or healthy level. Moreover, the disclosure provides a method of treating a diseased or dysregulated tissue, for example a tissue exhibiting the symptoms of a chronic inflammatory condition with potential to develop into a cancer; such conditions may include, for example, ulcerative colitis or pancreatitis. A method of this type may comprise administering microwave energy to modulate and/or restore the expression of one or more of the genes participating in key cancer pathways. This may prevent carcinogenesis.

Table 3 provides a list of the cancer pathway associated genes that can be modulated by the administration of microwave energy.

Table 3 below:

| | | Cancer pathway |
|---|---|---|
| Gene | Official full name | participation |
| KIT | Proto-Oncogene, Receptor | PI3K |
| | Tyrosine Kinase | RAS |
| GADD45B | Growth Arrest and DNA Damage | MAPK |
| | Inducible Beta | |
| Notch3 | suppressor of cytokine signaling 3 | Notch |
| CCND2 | Cyclin D2 | Wnt |
| | | Transcriptional |
| | | Regulation (KEGG) |
| | | JAK/STAT |
| | | PI3K |
| BAD | BCL2 Associated Agonist of Cell | PI3K |
| | Death | RAS |
| ID4 | Inhibitor of DNA Binding 4, HLH | TGF-B |
| | Protein | |
| WNT5A | Wnt Family Member 5A | Wnt |
| | | Hedgehog |
| RUNX1T1 | RUNX1 Partner Transcriptional | Transcriptional |
| | Co-Repressor 1 | Regulation (KEGG): |
| AKT3 | Protein kinase B, PKB | MAPK |
| | | JAK-STAT |
| | | PI3K |
| | | RAS |

The genes identified in Table 3 may be referred to as pathway associated genes.

In view of the above, the disclosure provides:
a method of modulating the KIT gene;
a method of modulating the GADD45B gene;
a method of modulating the Notch3 gene;
a method of modulating the CCND2 gene;
a method of modulating the BAD gene;
a method of modulating the ID4 gene;
a method of modulating the WNT5A gene;
a method of modulating the RUNX1T1 gene; and
a method of modulating the AKT3 gene.

Moreover, the disclosure provides a method of treating or preventing a disease or condition caused by and/or associated with:
aberrant expression of the KIT gene;
aberrant expression of the GADD45B gene;
aberrant expression of the Notch3 gene;
aberrant expression of the CCND2 gene;
aberrant expression of the BAD gene;
aberrant expression of the ID4 gene;
aberrant expression of the WNT5A gene;
aberrant expression of the RUNX1T1 gene; and
aberrant expression of the AKT3 gene;
said method comprising contacting a subject in need of treatment, with microwave energy. The microwave energy may be administered to the subject at a dose sufficient to modulate the expression (i.e. up- or down-regulate and/or restore the expression) of the relevant gene.

Additionally, the disclosure provides
a method of modulating the PI3K pathway;
a method of modulating the RAS pathway;
a method of modulating the MAPK pathway;
a method of modulating the Notch pathway;
a method of modulating the Wnt pathway;
a method of modulating the Transcriptional Regulation (KEGG) pathway a method of modulating the JAK/STAT pathway;
a method of modulating the TGF-B pathway; and
a method of modulating the Hedgehog pathway.

Moreover, the disclosure provides a method of treating or preventing a disease or condition caused by and/or associated with:
aberrant expression of the PI3K pathway;
aberrant expression of the RAS pathway;
aberrant expression of the MAPK pathway;
aberrant expression of the Notch pathway;
aberrant expression of the Wnt pathway;
aberrant expression of the Transcriptional Regulation (KEGG) pathway
aberrant expression of the JAK/STAT pathway;
aberrant expression of the TGF-B pathway; and
aberrant expression of the Hedgehog pathway;
said method comprising contacting a subject in need of treatment, with microwave energy. The microwave energy may be administered to the subject at a dose sufficient to modulate the expression (i.e. up- or down-regulate and/or restore the expression) the relevant pathway.

It should be understood that where a particular disease or condition is associated with the upregulation of a particular pathway, microwave energy may be used to downregulate the expression of that pathway.

Conversely, where a particular disease or condition is associated with the downregulation of a particular pathway, microwave energy may be used to upregulate the expression of that pathway.

Additionally or alternatively where a particular disease or condition is associated with the dysregulation of a particular pathway, microwave energy may be used to restore the expression of that pathway.

It should be noted (and without wishing to be bound by theory), microwave energy may be used to modulate the expression, function or activity of any given pathway because it has a modulatory effect on the expression of one or more pathway associated genes listed above.

The microwave energy may be supplied by a microwave generator and administered to a subject at a frequency of between about 300 MHz and about 300 GHz. In one teaching the microwave energy may be administered to a subject at a frequency of between about 900 MHz and about 200 GHz. In another teaching, the microwave energy may be administered to a subject at a frequency of between about 900 MHz and about 15 GHz. By way of example, the microwave energy may be administered (via a microwave energy generator) at about 2.45 GHZ, about 5.8 GHz about 6 GHZ, about 7 GHZ, about 7.5 GHZ, about 8 GHz, about 8.5 GHZ (for example from about 7.5 GHZ-about 8.5 GHZ), about 9 GHZ, about 10 GHz, about 11 GHz, about 12 GHZ, about 13 GHz or about 14 GHz. The microwave frequency may be administered at a frequency sufficient to have a therapeutic effect but not to affect a healthy tissue. By way of example, the microwave energy may be administered at 8 GHz—this may treat a diseases tissue but may not penetrate into a healthy tissue and/or beyond a depth of about 5 mm.

The microwave treatment may be minimally invasive.

The microwave treatment may be non-thermal, mild-hyperthermic or sub-ablative in nature.

The microwave treatment may provide a sub-ablative thermal stimulus.

The microwave treatment may not cause tissue destruction or necrosis.

The microwave treatment may be ablative.

The microwave treatment may comprise microwave energy which is ‘non-ablative’, ‘mildly ablative’, or ablative. A ‘non-ablative’ treatment may comprise only a treatment duration—perhaps, for example a treatment duration of about 1-5s or more. A ‘non-ablative’ treatment might comprise the use of microwave energy at a very low energy level energy such as 10-50 J or more, so as to cause no direct tissue or skin damage. Without wishing to be bound by theory, a ‘non-ablative’ treatment may use or exploit non-destructive thermal mechanisms (high electric fields, interruption or modulation of intracellular signalling/ion channels).

A ‘mildly ablative’ treatment with microwave energy may comprise a treatment duration of about 2-8s or more. The total amount of energy used may be low such as 30-80 J so as to cause no direct damage and only a mild to moderate elevation of temperature. A mildly-ablative treatment may produce modest thermal effects (heat shock, DAMPs and NOS elevation/expression, mild inflammation etc.) and promote apoptosis within (or of) treated tissue.

An ‘ablative’ treatment comprises the use of a moderate to higher level of microwave energy such as 50-100 J or more. The microwave energy may be used for a prolonged duration of around 3-10s or more. This may result in some direct tissue damage, a moderate to high level of temperature elevation (within the treated tissue) and potentially some direct tissue damage/necrosis.

It should be noted that the specifics of a useful dose may vary depending on the gene(s) to be modulated, the subject (age, weight, condition, history etc.), tissue or organ to be treated and the disease or condition to be treated and/or prevented. One of skill will be able to tweak any aspect of the microwave energy dose to fit the clinical circumstances including the effects of concomitant therapies and specific combined therapies. In this regard, a microwave-based therapy as described herein may be combined or administered together with another drug or therapeutic strategy. Microwave energy may be administered before, concurrently with or after any other type of therapy.

Microwave energy for use in the various methods described herein can comprise an input power of 0.1 W to 50 W. For example the microwave energy may be delivered at a power of about 1 W, about 2 W, about 5 W, about 8 W, about 10 W, about 15 W, about 20 W, about 25 W, about 30 W, about 40, about 50 W. The microwave energy may be administered at a single fixed power or at a range of different powers. The microwave energy may administered at a plurality of different powers. In one teaching, the microwave energy to be administered may be adjusted between different powers in increments of 0.1 W.

The input power may be applied for a duration of anywhere between about 0.1 s to 30 minutes. For example, the microwave may be applied for anywhere between about 1s, 2s, 5 s, 10 s, 30 s, 60 s to about 1 min, 2 min, 5 min or 10 min.

The microwave energy may be administered as a repeat dose. For example, the microwave energy may be administered to a subject as 5 individual doses of microwave energy. The duration of each dose may be the same or different. For example, each dose may last 5 s. A user may pause between administering each dose. The duration of the pause between each dose may be the same or different with a 20 s pause in between each treatment. The pause refers to disabling microwaves emission from the generator, preferably controlled using user interface. The microwave treatment can be applied for anywhere between about 2 times or 5 times or 10 times or 15 times, typically between 3-6 times. The pause can be anywhere between about 1 s, 5 s, 10 s, 20 s, 60 s, typically between 5 s-20 s. The dose may be administered as part of a treatment regimen made up of many dose deliveries with days, weeks or months between them.

Microwave energy may be used to deliver or administer a thermal effect to a subject or to a tissue thereof. For example, microwave energy may be used to raise the temperature in a subject (or a tissue thereof) from a first temperature to a second temperature. The first temperature may be equal to the temperature of the untreated subject/tissue. The second temperature may be higher than the first temperature. For example, the first temperature may be about 35° C., 36° C., 37° C. 38° C., 39° C. or 40° C.; the second temperature may be about 39° C., 40° C., 41° C., 42° C., 43° C., 44° C., 45° C., 46° C., 47° C., 48° C., 49° ° C., 50° C., 51° C., 52° C., 53° C., 54° C., 55° C., 56° C., 57° C., 58° C. or 59° C., 60° C. or 61° C. In one teaching the second temperature may not reach 60 ºC. Microwave energy may be used to raise the temperature in a subject or a tissue thereof of, from about 37° C. to about 59° C. For example, microwave energy may be administered to a subject so as to raise a temperature in that subject (or a tissue thereof) from about 42° C. and about 48° C.

In one teaching, the temperature may be raised from a first temperature to two or more different temperatures. The actual temperature rises will vary depending on, for example, the severity and complexity of the disease.

In one teaching, the second temperature may be maintained (to with an accuracy of about +/−0.5)° ° C. For example, once the desired second temperature has been reached, that temperature may be maintained for anywhere between about 1s and 30 min. For example, the second temperature may be maintained for about 1 min, 5 min, 10 min, 15 min, 20 min or 25 min.

By way of example, the temperature of a tissue to be treated may be raised from a first temperature (for example a first temperature equal to the temperature of the tissue before application of any microwave based treatment) and kept constant within +/−0.5° C. to any suitable second temperature, for example 43 ºC for the entire duration of the treatment, for example for 10 min.

Microwave energy may be administered as a series of alternate high and low microwave power pulses or as a dose intended to raise and maintain the temperature of the target tissue within +/−0.5° C. For example, an initial high dose may be used to elevate the temperature of the tissue from a first temperature (for example a temperature equal to the temperature of the untreated tissue) to a second, higher temperature. A lower dose of microwave energy (with a lower power rating that is used in the first dose) may then be administered in order to maintain that second temperature in the tissue. The second temperature may be maintained for any suitable time. Indeed, the temperature rise in the tissue can be controlled and maintained to avoid tissue necrosis and to induce mechanisms and processes related to any of the genes described herein, immune modulation events and/or the resetting of dysregulated, aberrantly expressed and/or cross-linked pathways.

By way of example, the microwave energy may be administered at 20 W for 10 s to raise the temperature of the tissue from a first temperature (perhaps a first temperature equal to the temperature of the untreated tissue) to a second temperature, for example to 43° C. This may then be followed by another lower dose of about, for example, 2 W to maintain the temperature of the tissue to 43° C. The lower dose may be maintained for as long as it is intended to maintain the second temperature within the tissue. For example, the second dose may be applied for up to about 300, 400, 500, 600, 700 seconds or longer.

The methods may be applied to biopsies, samples (provided by or obtained from a subject) and in vitro. Accordingly, the disclosure provides an in vitro method or use of modulating the expression of one or more genes, said method or use comprising exposing a tissue to microwave energy (at any dose or amount as described herein).

Any of the methods described herein may be applied or administered to a human or animal subject and to any tissue, organ or region thereof. A method (or microwave energy) may be applied or administered to a tissue, organ or region in a subject of any geometry and anywhere in the body. A human or animal subject to be treated may be predisposed and/or susceptible to, for example, an inflammatory disease or condition. A tissue, organ or region to be administered a microwave energy based treatment (as described herein) may exhibit signs and/or symptoms of an inflammatory condition.

Any of the microwave-based methods described herein may be applied or administered to a diseased tissue. A diseased tissue may be any tissue exhibiting the signs or symptoms characteristic of one or more diseases. Without wishing to be bound by theory, a diseased tissue may be administered a microwave-based treatment for the purpose of modulating the expression of one or more genes within that tissue. The genes to be modulated may be any one or more of those described herein and/or may be associated with a specific disease and/or condition to be prevented or treated.

In one teaching, the term tissue may embrace epithelial tissue. The term “diseased tissue” may embrace a diseased epithelial tissue. The terms ‘treated tissue’ may relate to tissue that has been administered a microwave treatment of this disclosure.

Any reference to normal or healthy tissue is a reference to tissue which does not exhibit any signs or symptoms of a disease or condition; is not wounded or damaged and/or does not contain genes or cellular pathways which are aberrantly expressed. A tissue to be administered a microwave-based method of this may comprise skin or diseased skin. Diseased skin may exhibit the signs or symptoms characteristic of one or more diseases and/or conditions associated with the skin. Skin which may benefit from treatment using microwave energy may include, for example, inflamed skin, injured (breached, torn or cut) skin. Microwave energy may also be applied to the skin with one or more scars, erosion and/or lesions.

Without wishing to be bound by theory, it is suggested that following exposure to microwave energy, one or more genes within the tissue (including, for example skin) may be modulated such that some aspect of a disease or condition (for example one or more symptoms) is/are improved or resolved. In other words, microwave energy may be used to modulate the expression of one or more genes to resolve or improve one or more symptom(s) or features which are characteristic of the disease or condition.

A tissue to be administered a microwave-based method of this disclosure may be derived, provided or obtained by/from a subject to be treated using a method described herein. Accordingly, the tissue may be an in-situ tissue, in vivo tissue or a biopsy of ex vivo sample.

Based on its ability to modulate the expression of a number of genes, microwave energy (as described herein) may be applied to the treatment and/or prevention of a number of disease and/or conditions—especially those characterised by aberrant and/or defective gene expression. It should be noted that aberrant and/or defective gene expression may be determined relative to the gene expression in a normal or healthy tissue (that is, a tissue which does not exhibit the signs and/or symptoms of a disease associated with aberrant and/or defective gene expression).

In some cases, a disease or condition may be caused by an increase in the expression of one or more genes. In such circumstances, microwave energy may be used to normalise gene expression—by, for example, suppressing, inhibiting and/or reducing the expression of any over-expressed gene. This helps restore the levels of expression to, or close to, normal levels.

In other cases, a disease or condition may be caused by a decrease in the expression of one or more genes. In such circumstances, microwave energy may be used to normalise gene expression—by, for example, promoting, increasing, stimulating and/or enhancing the expression of any under-expressed gene. This helps restore the levels of expression to, or close to, normal levels.

The present disclosure provides a method of treating a disease or condition characterised and/or caused by the dysregulation of one or more genes in a tissue. In some cases, diseased or dysregulated tissue exhibits abnormally lower expression of certain genes when compared to a healthy or normal form of the same tissue. Treatment with microwave energy can restore or normalize (by, for example upregulation) the expression of those genes in the diseased or dysregulated tissue where the expression of the said genes is upregulated (induced, promoted or stimulated) and restored or normalized. In other cases, diseased or dysregulated tissue exhibits abnormally higher expression of certain genes when compared to a healthy form of the same tissue. In those cases, administration of microwave energy can downregulate (suppress, inhibit or reduce) and restore or normalize (by, for example, downregulation) expression of the relevant genes.

Without wishing to be bound by theory, the modulation (for example, restoration and/or normalization) of gene expression in a tissue, for example a diseased tissue may treat and/or prevent diseases and/or conditions characterised by inflammation and/or uncontrolled cell growth/differentiation. By way of non-limiting example, the effect of microwave energy on the various genes disclosed herein, may be applied to the prevention and treatment of a carcinoma which develops as a consequence of chronic inflammation.

A method of treating a disease or condition characterised and/or caused by the dysregulation of one or more genes in a tissue may comprise administering a subject in need thereof, microwave energy to restore and/or normalize gene expression in the tissue.

In some cases, a diseased or dysregulated tissue exhibits abnormally lower expression of certain genes when compared to the healthy form of the same tissue. In those cases, upon treated with the said microwave system the expression of those same genes in the diseased or dysregulated tissue is upregulated (induced, promoted or stimulated) and restored or normalized.

The present disclosure provides a method of treating a disease or condition characterised and/or caused by dysregulated intrinsic and/or extrinsic pathways. In such methods, the administration of microwave energy (as described herein) may rectify and reset dysregulated intrinsic and/or extrinsic pathways. Rectification and resetting of any dysregulated intrinsic and/or extrinsic pathways may occur via the modulatory effect of microwave energy on the expression of one or more of the genes associated with the dysregulated intrinsic and/or extrinsic pathways.

The present disclosure provides a method of treating a disease or condition characterised and/or caused by aberrant and/or defective homeostasis. In such methods, the administration of microwave energy (as described herein) may rectify and restore any aberrant, dysregulated and/or defective homeostasis event. Rectification and restoration of any dysregulated and/or defective homeostasis event may occur via the modulatory effect of microwave energy on the expression of one or more of the genes associated with the dysregulated and/or defective homeostasis event.

This disclosure also provides a method of restoring gene expression in dysregulated tissue, including, for example an epithelial tissue. In such methods, the administration of microwave energy (as described herein) may rectify any aberrant, dysregulated or defective gene expression in a tissue (for example an epithelial tissue). Rectification of any aberrant or defective gene expression in a tissue (for example an epithelial tissue) may occur via the modulatory effect of microwave energy on the expression of one or more of the genes which are being aberrantly of defectively expressed in the tissue (for example, epithelial tissue).

Additionally, the disclosure provides a method of stimulating, promoting and/or enhancing tissue repair, healing and/or regeneration. In such methods, the administration of microwave energy (as described herein) may stimulate, promote and/or enhance tissue repair, healing and/or regeneration. The stimulation, promotion and/or enhancement of tissue repair, healing and/or regeneration may occur via the modulatory effect of microwave energy on the expression of one or more of the genes associated with the stimulation, promotion and/or enhancement of tissue repair, healing and/or regeneration.

In view of the above, one of skill will appreciate that where a disease or condition is known to be associated with a level of expression of a particular gene, microwave energy may represent a novel route to the treatment and/or prevention of that disease or condition. By way of example, microwave energy may be used to restore the aberrant expression of the one or more genes that is known to be associated with the disease or condition.

Tissue to be exposed to microwave energy (for the purpose of modulation the expression of one or more genes within that tissue) may comprise a diseased or dysregulated tissue (e.g. a tissue harboring aberrantly (under and/or over) expressed genes), for example an epithelial tissue.

A tissue to be treated or administered microwave energy may be any tissue exhibiting the signs or symptoms characteristic of one or more diseases such as chronic inflammation.

A ‘diseased’ tissue may have the potential to cause carcinogenesis and/or can be predisposed or susceptible to a precancerous condition.

A tissue to be administered microwave energy may be an epithelial tissue. Accordingly, a tissue to be administered microwave energy may comprise a diseased or dysregulated epithelial tissue. Said tissue may be exhibiting the signs or symptoms characteristic of one or more diseases, for example an inflammatory condition.

A tissue (for example an epithelial tissue) to be administered microwave energy may form lining of range of tissues such as:
a. Simple squamous epithelium: Blood and lymph vessels, air sacs of lung, lining of heart
b. Simple cuboidal epithelium: Kidney tubules, pancreas, salivary glands, thyroid glands
c. Simple columnar epithelium: GI tract organs stomach, small intestine, colon, uterus
d. Pseudostratified epithelium: Upper respiratory tract, uterine tubes
e. Stratified squamous epithelium
   a. Non-keratinized: Oral, esophagus, larynx, vagina, anus
   b. Keratinized: Skin
f. Stratified cuboidal epithelium: Sweat, excretory, mammary glands
g. Stratified columnar epithelium: Male urethra, sensory organs
h. Transitional epithelium: Distendable organs such as bladder, ureters etc.
i. Germinal simple squamous-to-cuboidal epithelium: Ovary

A subject to be treated using a method described herein may be suffering from or predisposed/susceptible to, one or more acute and/or chronic inflammatory conditions.

A method of this disclosure may induce beneficial thermal and non-thermal effects.

By way of example and in one teaching, any of the methods described herein may be used to treat and/or prevent conditions occurring in or on a tissue comprising columnar epithelium, for example the tissues of the GI (gastrointestinal) tract.

Conditions and/or diseases which may be treated, improved and/or prevented by the administration of microwave energy may include, for example, inflammatory bowel diseases (IBD) including Crohn disease, and ulcerative colitis, irritable bowel syndrome, short bowel syndrome, diverticulitis, gastroenteritis and peptic ulcers. A microwave-based method of this disclosure may treat, improve and/or prevent any of these conditions by restoring and/or normalizing any genes which have become dysregulated (up or down-regulated) and which are associated with or are markers of these diseases.

Any of the microwave-based methods described herein may be used to treat or prevent chronic inflammation within a tissue and to reduce the risk of an associated metastatic carcinoma. By way of example, chronic GI inflammation may lead to colorectal cancer; the application of microwave energy to control the initial inflammatory events (by gene modulation) may in turn reduce the risk of colorectal cancer. Without wishing to be bound by theory, the use of microwave energy to normalize any aberrantly expressed and/or dysregulated genes may restore tissue homeostasis and rectify tissue dysregulation; this may re-set any dysregulated intrinsic and extrinsic pathways promoting tissue repair, tissue healing and/or tissue regeneration.

In another teaching, the microwave energy may be used to treat and/or prevent diseases or conditions which affect simple cuboidal epithelium, for example, the pancreatic epithelium lining. Such diseases or conditions may include e.g. acute and chronic inflammation of the pancreatic epithelium such as pancreatitis which may lead to pancreatic cancer. Again, and without wishing to be bound by theory, a microwave-based treatment may treat or prevent diseases or conditions of this type via the restoration of tissue homeostasis through the modulation (e.g. normalizing/resetting) of any dysregulated intrinsic and extrinsic genes pathways to promote tissue repair, healing and regeneration.

In other teachings, the microwave-based methods of this disclosure may be used to treat and/or prevent diseases/conditions in tissues which comprise translational epithelium.

Such disease and/or conditions my include, for example, acute and chronic inflammation of the urothelial epithelium lining in the urinary bladder leading to squamous cell carcinoma of the bladder lining and metastatic bladder cancer. Again, and without wishing to be bound by theory, a microwave-based treatment may treat or prevent diseases or conditions of this type via the restoration of tissue homeostasis through the modulation (e.g. normalizing/resetting) of any dysregulated intrinsic and extrinsic genes pathways to promote tissue repair, healing and regeneration.

A microwave-based method of this disclosure may also be used to treat or prevent a disease or condition in a tissue comprising a germinal epithelial layer comprising more than one type of epithelium for example, simple squamous-to-cuboidal epithelium in the ovary. Diseases or conditions of this type may comprise acute or chronic ovarian epithelial inflammation leading to ovarian cancer. Without wishing to be bound by theory, a microwave-based treatment may treat or prevent diseases or conditions of this type via the restoration of tissue homeostasis through the modulation (e.g. normalizing/resetting) of any dysregulated intrinsic and extrinsic genes pathways to promote tissue repair, healing and regeneration.

In another embodiment, the methods and system disclosed here are administered for the treatment and/or prevention of diseases/conditions in the tissues comprising non-keratinized stratified squamous epithelium. Tissue of this type is found in the oral cavity, in oral mucosa and/or genital tissues. Diseases and/or conditions of this type may comprise one or more inflammatory diseases such as lichen planus, actinic cheilitis, cervical intraepithelial neoplasia, vaginal intraepithelial neoplasia, vulvar intraepithelial neoplasia which may lead squamous cell carcinoma. Without wishing to be bound by theory, a microwave-based treatment may treat or prevent diseases or conditions of this type via the restoration of tissue homeostasis through the modulation (e.g. normalizing/resetting) of any dysregulated intrinsic and extrinsic genes pathways to promote tissue repair, healing and regeneration.

A microwave-based method of this disclosure may be administered to the skin and/or to any tissue comprising keratinized stratified squamous epithelium. Microwave energy may be administered for the treatment and/or prevention of persistent inflammation of the skin tissue such as, for example, psoriasis or atopic dermatitis. The disclosed methods may also be used to prevent chronic skin inflammation from developing into squamous cell carcinoma (SCC) and basal cell carcinoma (BCC). Further, the disclosed methods may be used in treating epithelial carcinomas such as SCC and BCC. Without wishing to be bound by theory, a microwave-based treatment may treat or prevent diseases or conditions of this type via the restoration of tissue homeostasis through the modulation (e.g. normalizing/resetting) of any dysregulated intrinsic and extrinsic genes pathways to promote tissue repair, healing and regeneration.

In another teaching, a microwave-based method of this disclosure may be administered to facilitate wound healing. For example, applying the methods described herein on a wound that has occurred anywhere in the body such as skin or colon. Wound healing comprises 4 key stages viz. Homeostasis, Inflammation, Proliferation phase and Maturation. While each phase is essential, quite often an imbalance in one of them can cause chronic or persistent wound. For example, inflammation phase is crucial as it protects against excessive bleeding and infection at the wound site. However, it causes a severe tissue damage if it is prolonged or excessive, leading to chronic inflammation indicating dysfunctional immune function. The present microwave method system can be used to control or eliminate probability of excessive or persistent chronic inflammation thereby enhancing the tissue repair, healing and regeneration. The said method can aid and accelerate the proliferative phase of the wound healing to help rebuild and restore the tissue.

Any of the methods described may be used to modulate and/or normalize gene expression of the immunomodulatory biomarkers in diseased and/or dysregulated tissue. These immunomodulatory markers may participate in, for example, key immunomodulatory pathways that promote the various stages of the healing process. The microwave-based methods described herein may target key immunomodulatory pathways to ameliorate a disease and/or condition. Immunomodulatory markers may also participate in key cancer pathways. The methods described herein may target key cancer pathways to prevent progression into carcinogenesis. Without being bound by theory, microwave energy may act to rectify tissue dysregulation and reset dysregulated intrinsic and extrinsic pathways to restore tissue homeostasis and promote tissue repair, healing and regeneration.

A gene or genes to be modulated by microwave energy may be directly or indirectly associated with a disease or condition affecting one or more tissue(s). For example, one or more of the genes may be involved with one or more immunomodulatory or cancer pathways or mechanisms associated with a disease or condition of an epithelial tissue.

The modulated gene(s) may encode or provide factors associated with the host immune system. For example, the modulated gene(s) may encode or provide factors which are immunomodulatory.

Additionally, or alternatively, the modulated gene(s) may be classified as “cancer” or “oncogenic” genes—that is to say, their expression is associated with one or more types of cancer.

Without wishing to be bound by theory, the application of microwave energy to a tissue may induce beneficial thermal effects within said tissue. These effects may be induced locally or to a wider region within the tissue. The application of microwave energy may induce beneficial non-thermal effects such as but not limited to, dielectrophoretic effects, electrophoresis effects, electroosmosis effects, electroporation effects, high frequency (GHz) mechanical resonance effects (relating to fracturing viral particles), enhancement of protein reaction rates, optimized immunomodulatory signaling, improved enzyme stability, improved cellular uptake and cellular function of cell and homogeneous orientation of large molecules.

### DETAILED DESCRIPTION

The present invention will now be described with reference to the following figures which show:

The present invention will now be described with reference to the following figures which show:

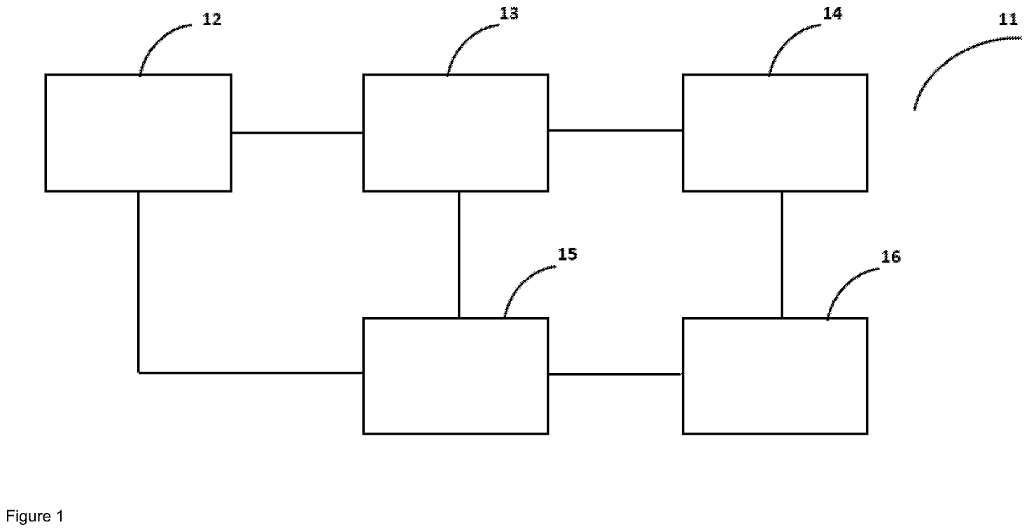

FIG. 1: A schematic illustration of a microwave treatment system, in accordance with embodiments.

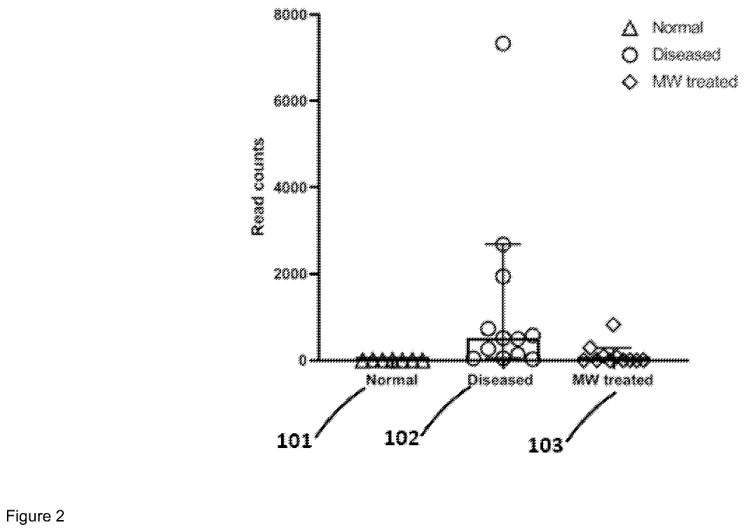

FIG. 2: Example of IL8 gene count in normal, diseased and microwave treated and epithelial tissue.

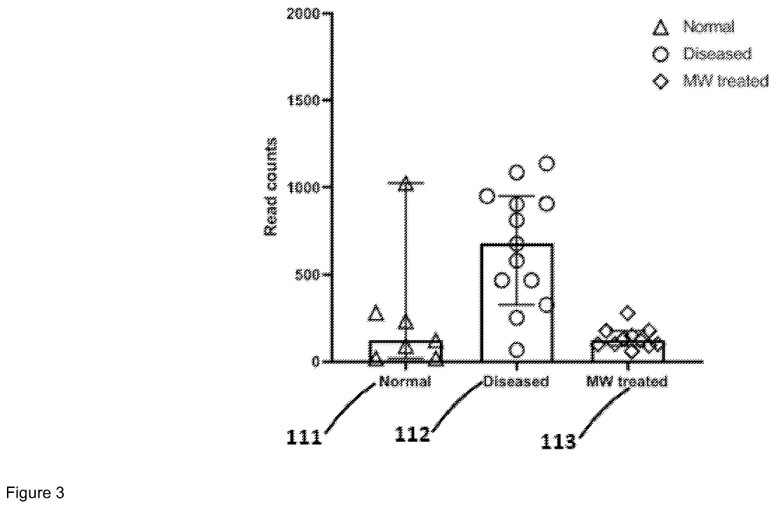

FIG. 3: Example of SOCS3 gene count in normal, diseased and microwave treated and epithelial tissue.

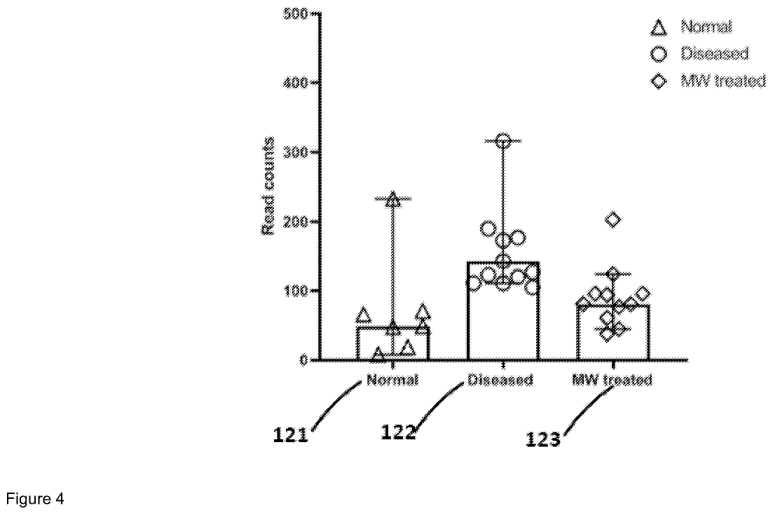

FIG. 4: Example of EGR-1 gene count in normal, diseased and microwave treated and epithelial tissue.

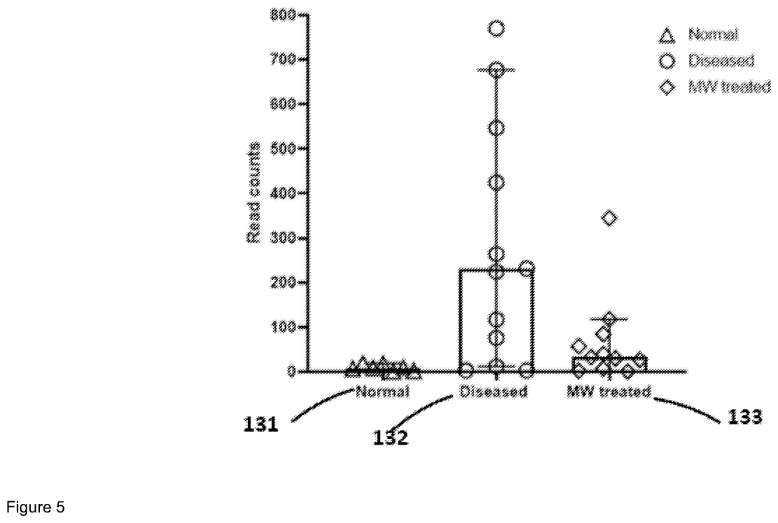

FIG. 5: Example of CD79A gene count in normal, diseased and microwave treated and epithelial tissue.

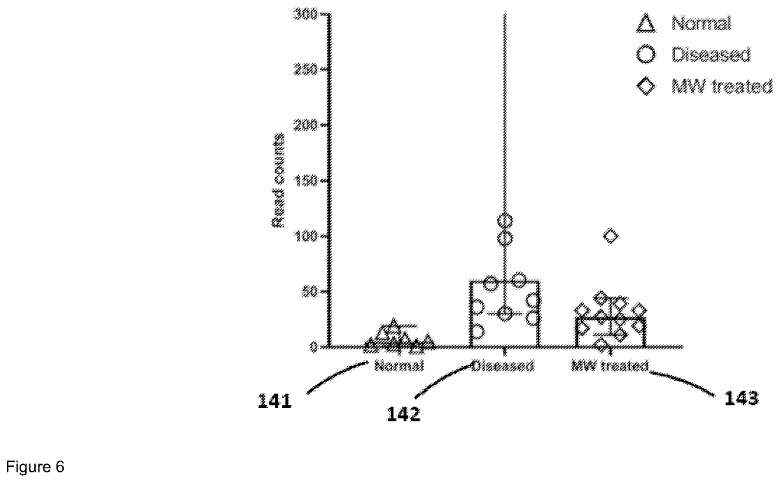

FIG. 6: Example of IL1B gene count in normal, diseased and microwave treated and epithelial tissue.

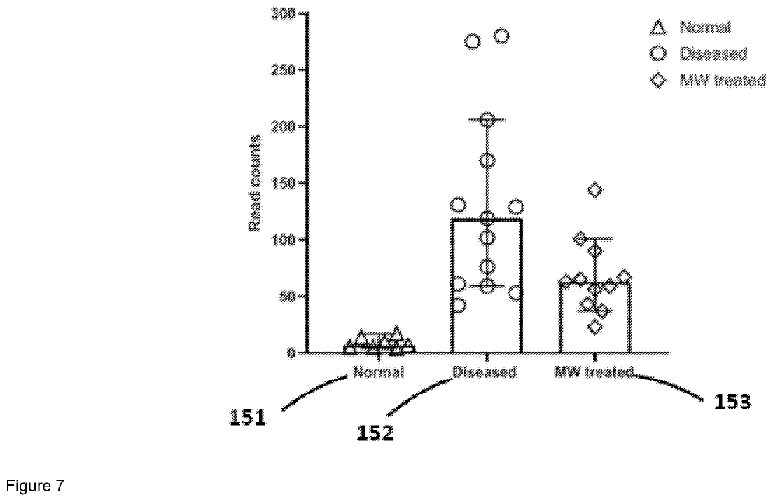

FIG. 7: Example of TNFRSF13C gene count in normal, diseased and microwave treated and epithelial tissue.

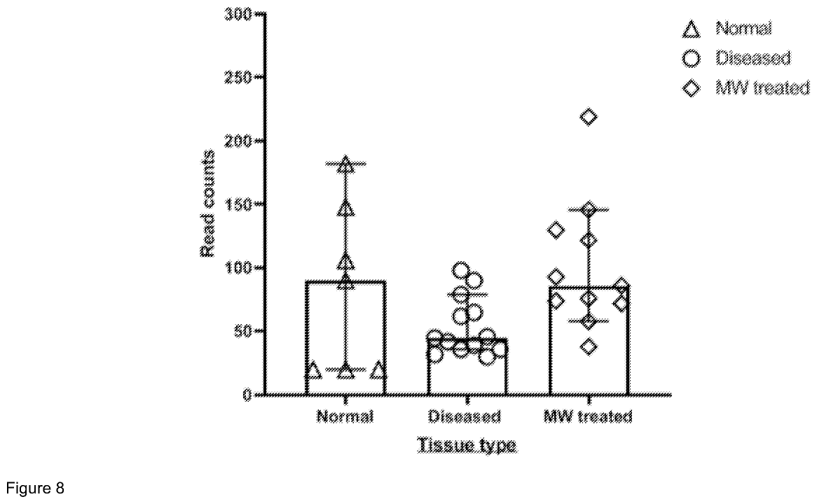

FIG. 8: Example of a gene count participating in PI3K pathway for normal, diseased and microwave treated and epithelial tissue.

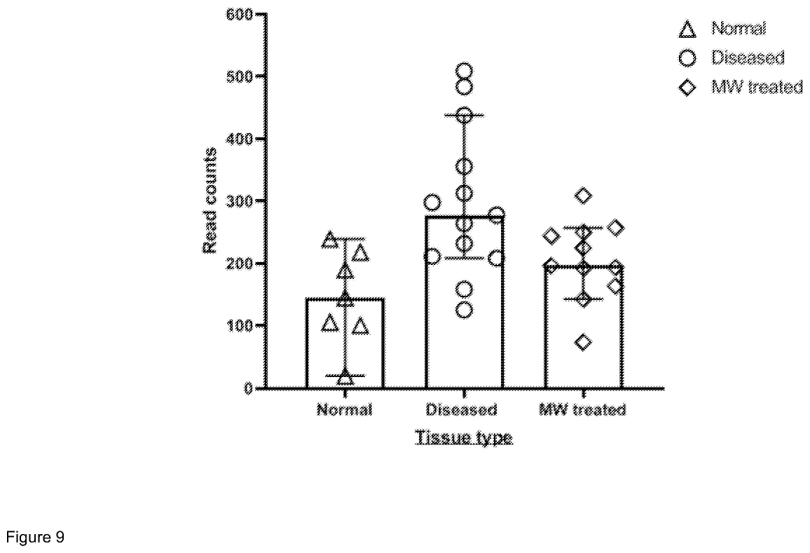

FIG. 9: Example of a gene count participating in MAPK pathway for normal, diseased and microwave treated and epithelial tissue.

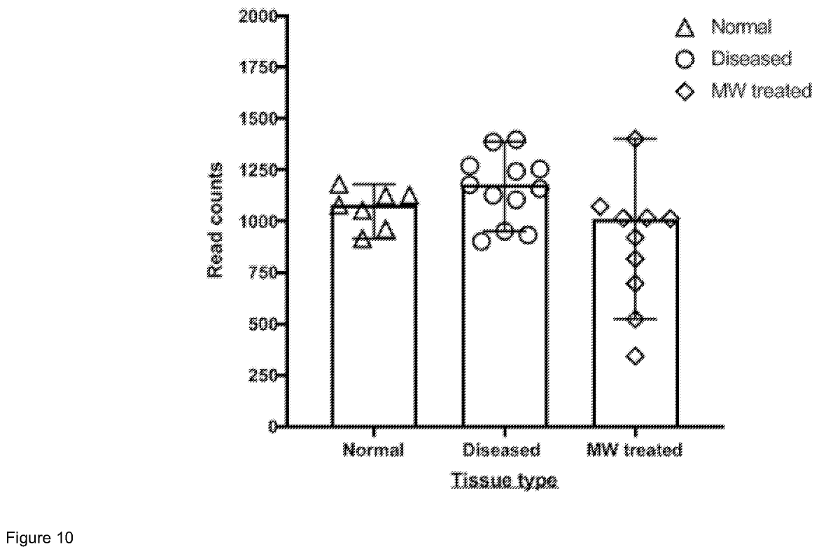

FIG. 10: Example of a gene count participating in Notch pathway for normal, diseased and microwave treated and epithelial tissue.

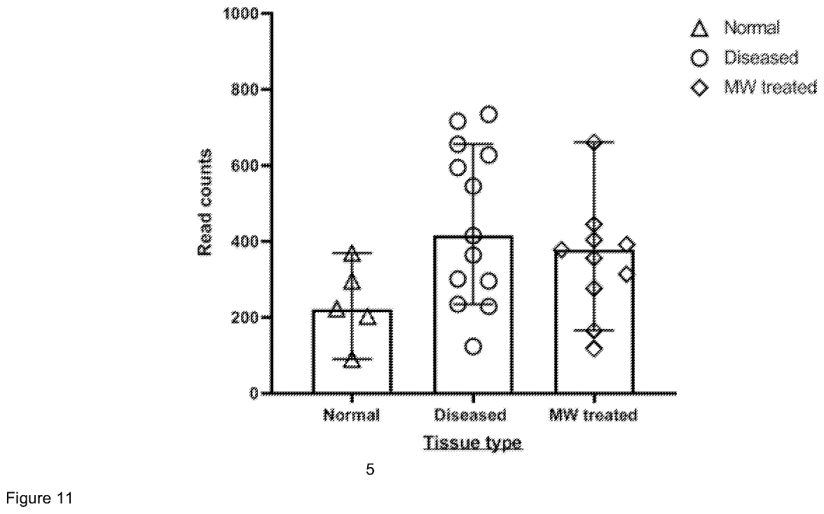

FIG. 11: Example of a gene count participating in Wnt pathway for normal, diseased and microwave treated and epithelial tissue.

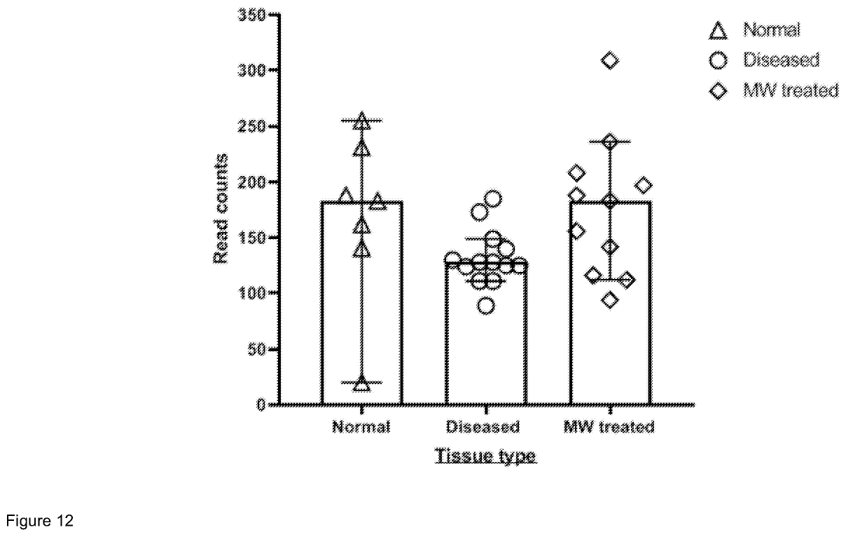

FIG. 12: Example of a gene count participating in RAS pathway for normal, diseased and microwave treated and epithelial tissue.

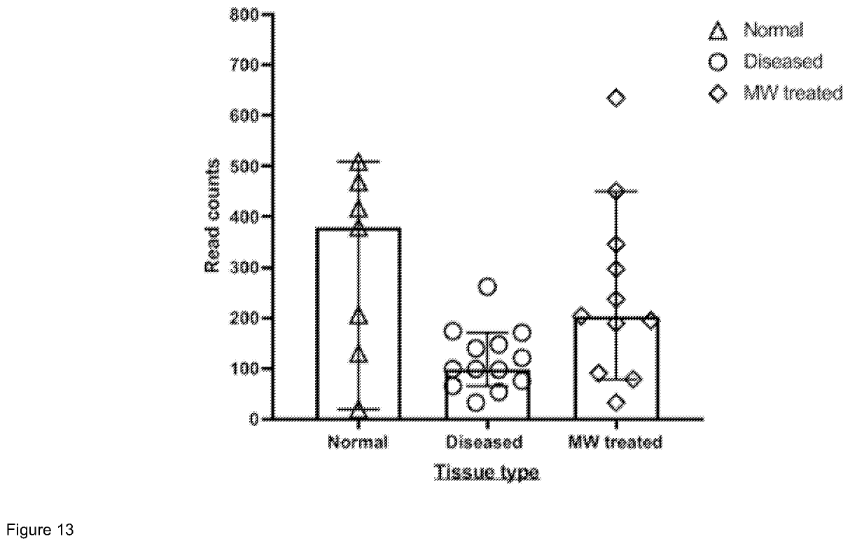

FIG. 13: Example of a gene count participating in TGFb pathway for normal, diseased and microwave treated and epithelial tissue.

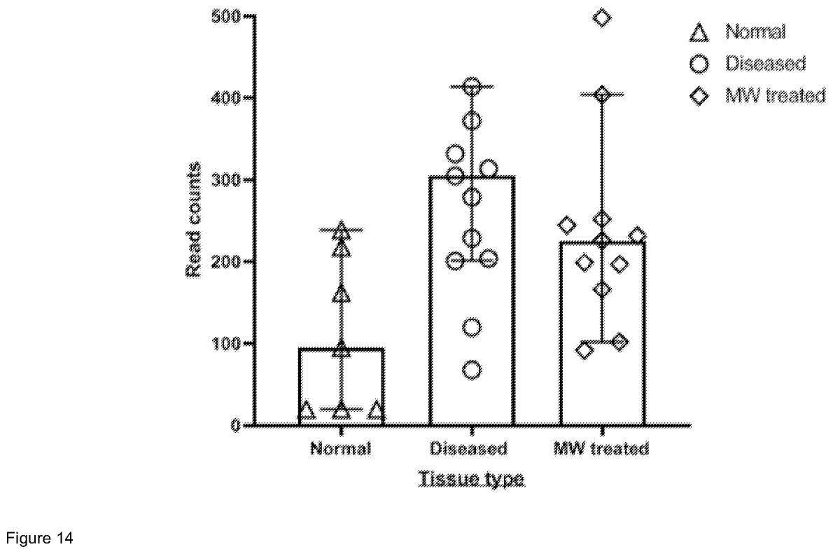

FIG. 14: Example of a gene count participating in Hedgehog pathway for normal, diseased and microwave treated and epithelial tissue.

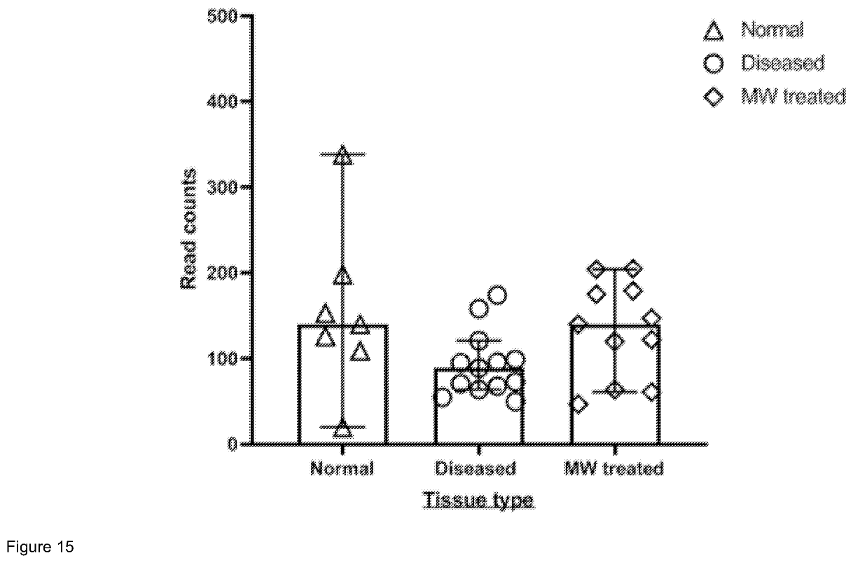

FIG. 15: Example of a gene count participating in Transcriptional Regulation (KEGG) for pathway normal, diseased and microwave treated and epithelial tissue.

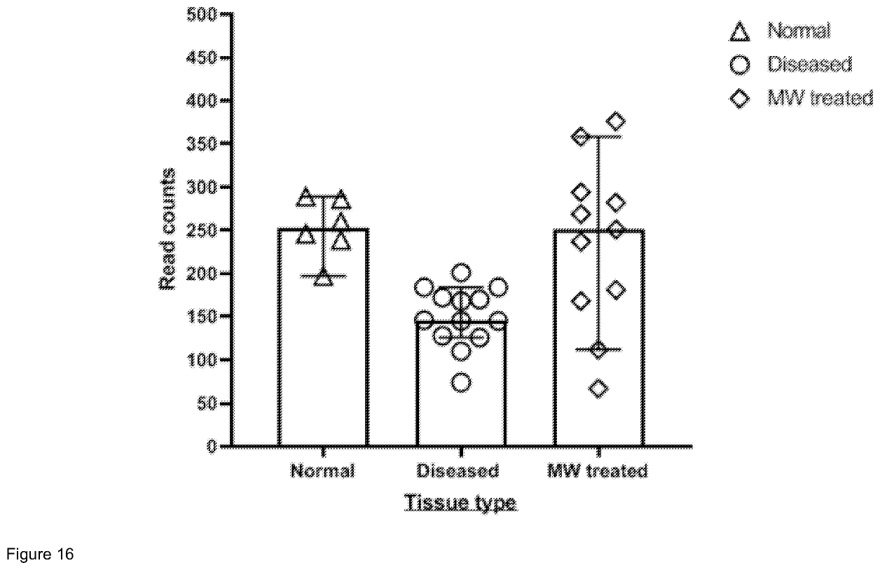

FIG. 16: Example of a gene count participating in JAK-STAT pathway for normal, diseased and microwave treated and epithelial tissue.

### DETAILED DESCRIPTION OF THE DRAWINGS

In FIG. 1, a typical microwave treatment system 11 in accordance with embodiments is illustrated. The system comprises a microwave source 12 for providing microwave energy. The microwave source 12 is connected to auxiliary microwave components 15 such as energy conduit cable and to the microwave applicator 16. The microwave applicator 16 relates to a microwave antenna. Further, the microwave treatment system 11 uses a system controller 13 that is used to control at least one property of the microwave radiation provided by the source 12. For example, system controller 13 may allow the user to modulate and optimize the power, time, frequency, wavelength and/or amplitude of the microwave energy. The system 11 further consists a monitoring system 14 for monitoring the delivery of energy such that to maintain the effective microwave emission delivery into the tissue.

Microwave energy for use according to this disclosure may be applied at a frequency of between about 300 MHz and about 300 GHz. In some embodiments, the frequency of the microwave energy may range from between about 900 MHz and about 15 GHz and preferably about 2.45 GHZ, about 5.8 GHz about 6 GHZ, about 7 GHZ, about 7.5 GHZ, about 8 GHZ, about 8.5 GHZ (for example from about 7.5 GHZ-about 8.5 GHZ), about 9 GHz, about 10 GHz, about 11 GHZ, about 12 GHZ, about 13 GHz or about 14 GHz. The microwave frequency according to the embodiments in this disclosure may be high enough, for example 8.0 GHz to restrict the microwave energy travelling further in the healthy tissue, for example less than 5 mm.

The microwave energy in accordance with the embodiments may be delivered at a power of anywhere between about 0.1 W and about 50 W. For example, the microwave energy may be delivered at a power of about 1 W, about 2 W, about 5 W, about 8 W, about 10 W, about 15 W, about 20 W, about 25 W, about 30 W, about 40, about 50 W. The microwave energy may be delivered in increments of 0.1 W. The microwave energy may be delivered at a single fixed power or at a range of different powers.

The microwave energy may be administered to raise the temperature of the target tissue for any suitable time including for anywhere between about 0.1 s and 30 min. For example, the microwave may be applied for anywhere between about 1s, 2s, 5 s, 10 s, 30 s, 60 s to about 1 min, 2 min, 5 min or 10 min. The microwave treatment may be administered as a repeated dose. For example, the microwaves can be applied for 5 times each lasting 5 s with a 20 s pause in between each treatment. The pause refers to disabling microwaves emission from the generator, preferably controlled using user interface. The microwave treatment can be applied for anywhere between about 2 times or 5 times or 10 times or 15 times, typically between 3-6 times. The pause can be anywhere between about 1 s, 5 s, 10 s, 20 s, 60 s, typically between 5 s-20 s.

The microwave radiation may be used to apply an energy to provide thermal effects such as raising the temperature of the diseased tissue section between about 37° C. to about 59° C. For example, the microwave energy may be applied to raise the temperature of the diseased tissue section of anywhere between about 42° C. and about 48° C. In some embodiments, alternatively two or more different temperatures may be exploited depending upon factors such as severity and complexity of the disease.

In some embodiments, temperature of the tissue may be raised and kept constant within +/−0.5° C. to any suitable temperature, for example 43 ºC for the entire duration of the treatment, for example for 10 min.

In some embodiments, microwave energy may be administered as a series of alternate high and low microwave power pulses or as a profile or treatment envelope to raise and maintain the temperature of the target tissue within +/−0.5° C., for example administering 20 W for 10 s to raise the temperature of the tissue to 43° C. followed by 2 W for 300 s to maintain the temperature of the tissue to 43° C.

Examples relating to the modulation of the gene count in the diseased, microwave treated and normal tissue achieved using the microwave system and method are presented herein. Here, normal refers to a healthy epithelial tissue (illustrated using triangular markers), diseased (illustrated using circular markers) refers to a diseased epithelial tissue whereas MW treated (illustrated using diamond shaped markers) refers to an epithelial tissue treated using presented microwave system and method. This disclosure provides further examples of genes participating in key cancer pathways and their restoration using the disclosed microwave energy techniques are presented herein.

In FIG. 2, IL8 (interleukin 8) gene count in the tissue, for example an epithelial tissue, before 102 and after microwave treatment 103 is shown and is compared with a normal tissue 101 for example healthy epithelial tissue. Microwave treated tissue restored the abnormally upregulated expression of the IL8 gene from the diseased epithelial tissue (count=510.5) and downregulate it (count=9) to be equivalent to the normal healthy epithelial tissue (count=7) statistically significant at 0.05. IL8 participates in numerous important immunomodulatory pathways such as Cytokine Signaling, Host-pathogen Interaction, Lymphocyte Activation, NLR signalling, Oxidative Stress, Th17 Differentiation, Th2 Differentiation, TNF Family Signaling and TLR Signaling. IL8 is a chemotactic cytokine (chemokines) and is a potent inflammatory chemoattractant molecule which has a central role in the augmentation and perpetuation of inflammation in gastrointestinal inflammation and malignancy. Further, IL8 plays an essential role in the transition of epithelial to a mesenchymal-like phenotype [epithelial-to-mesenchymal transition (EMT)] which is known to induce tumor cell motility and invasiveness, promoting metastasis of solid carcinomas [34].

Similarly, in FIG. 3, SOCS3 (Suppressor of cytokine signaling 3) is aberrantly upregulated in the diseased epithelial tissue 102 (count=679) as compared to the normal epithelial tissue 101 (count=123). Upon microwave treatment, the gene count is downregulated and restored 103 (count=123) statistically significant at 0.05. SOCS3 is an important immunological factor participating in Adaptive Immune System, Cytokine Signaling, Host-pathogen Interaction, MHC Class I Antigen Presentation, TNF Family Signaling, Type I Interferon Signaling, Type II Interferon Signaling. Further, SOCS is a key physiological regulator of cytokine-mediated STAT3 signaling which has been shown to play a major role in transmitting inflammatory cytokine signals to the nucleus. For example, STAT3 signaling links IL-22 signaling in intestinal epithelial cells to mucosal wound healing promoting epithelial repair [35].

Increased mRNA expressions of EGR1 (Early growth response proteins-1) have been observed to increase Transcription factors and are found elevated in epithelial inflammatory conditions [36]. In FIG. 4, using the system and method presented in this disclosure EGR-1 count in the diseased tissue is normalized at statistically significant level of 0.05. EGR-1 count is aberrantly upregulated in the diseased epithelial tissue 122 (count=143) as compared to the normal tissue 121 (count=49) which is restored upon microwave treatment 123 (count=81).

In FIG. 5, CD79A (Cluster of differentiation 79A also known as B-cell antigen receptor complex-associated protein alpha chain and MB-1 membrane glycoprotein) count in the diseased tissue is shown to be restored upon microwave treatment, statistically significant at 0.05. CD79A participates in various important immunomodulatory pathways such as Adaptive Immune System, B cell Receptor Signaling, Lymphocyte activation. CD79A has been found to be overexpressed in diseased epithelial samples of inflammatory conditions [37]. In FIG. 15, CD79A count in the in the diseased epithelial tissue 132 (count=231) is aberrantly upregulated as compared to the normal epithelial tissue 131 (count=33). Upon microwave treatment, the gene count is downregulated and restored 133 (count=9).

Inflammatory cytokines such as IL1B (interleukin 1, beta) are often upregulated in inflamed epithelial tissue implicating tissue damage and have been shown to correlate with epithelial disruption characterized by the mislocalization and reduced expression of tight junction proteins [38]. Elevated IL1B has also been widely implicated in regulating epithelial-to-mesenchymal transition memory phenotypes via epigenetic modifications in non-small cell lung cancer facilitating tumor progression [39]. IL1B participates in important immunomodulatory pathways such as Cytokine Signaling, Host-pathogen Interaction, Innate Immune System, Lymphocyte Activation, NF-kB Signaling, Th17 Differentiation, TNF Family Signaling, TLR Signaling. In FIG. 6, IL1B count in the in the diseased epithelial tissue 142 (count=60) is aberrantly upregulated as compared to the normal epithelial tissue 141 (count=5). Upon microwave treatment, the gene count is downregulated and restored 143 (count=27) at a statistically significant level of 0.05.

Tumor necrotizing factor (TNF), a pleiotropic cytokine is known to be another key regulator of cytokine production and has often been observed to be elevated in both the serum and mucosa of IBD patients [41]. Further, TNF family expressions are frequently detected in biopsies from cancers originating from by epithelial tumor cells, for instance ovarian and renal cancer [42]. TNFRSF13C in implicated to modulates key immunoregulatory pathways such as Cytokine Signaling, Host-pathogen Interaction, Lymphocyte Activation, NF-kB Signaling, TNF signaling. In FIG. 7, TNFRSF13C count in the in the diseased epithelial tissue 152 (count=7) is aberrantly upregulated as compared to the normal epithelial tissue 151 (count=119). Upon microwave treatment, the gene count is downregulated and restored 153 (count=63).

It is appreciated that although some of the genes have a smaller change in the expression when treated with microwave energy, this effect or magnitude of the change can be modulated by optimizing, for example increasing, the microwave energy dose. For example, by increasing the microwave dose by 20%-50%, it is possible to increase the effect of the microwave energy against the expression of any given gene.

Further, in FIG. 8 to FIG. 16 restoration of the genes (upon microwave treatment) participating in important cancer pathways is illustrated. These genes play a key role in the progression from chronic stage of the inflammation to the carcinogenesis and metastasis. For example, in FIG. 8 KIT (Proto-Oncogene, Receptor Tyrosine Kinase) which participates in PI3K pathway is abnormally downregulated in the diseased tissue as compared to the healthy tissue and is restored upon microwave treatment. Similarly FIG. 9 (GADD45B, Growth Arrest And DNA Damage Inducible Beta), FIG. 10 (Notch3), FIG. 11 (CCND2, Cyclin D2), FIG. 12 (BAD, BCL2 Associated Agonist Of Cell Death), FIG. 13 (ID4, Inhibitor Of DNA Binding 4, HLH Protein), FIG. 14 (WNT5A, Wnt Family Member 5A), FIG. 15 (RUNX1T1, RUNX1 Partner Transcriptional Co-Repressor 1), and FIG. 16 (AKT3, Protein kinase B, PKB) illustrate restoration of genes participating in key cancer pathways MAPK, NOTCH, APC (Wnt), RAS, TGFb, Hedgehog, Transcriptional Regulation (KEGG), and JAK-STAT respectively. Restoring genes participating in cancer pathways in diseased tissue for example may prevent such tissue from developing into malignant cancer.

A single gene participates in more than one cancer pathways for example, AKT3, Protein kinase B, PKB participates in PI3K, RAS, MAPK and JAK-STAT but for ease, each cancer pathway is shown using a single gene example.

For completeness, a brief description of the pathways linked to one or more of the microwave modulated gene(s) is described:
PI3K: The phosphatidylinositol 3′-kinase (PI3K)-Akt signaling pathway regulates fundamental cellular functions such as transcription, translation, proliferation, growth, apoptosis, protein synthesis, metabolism cell cycle and survival.
MAPK: The mitogen-activated protein kinase (MAPK) cascade is a highly conserved module that is involved in various cellular functions, including cell proliferation, differentiation and migration. Abnormal MAPK signaling may lead to increased or uncontrolled cell proliferation and resistance to apoptosis.
Notch: Intercellular signaling mechanism essential for proper embryonic development. The Notch proteins are single-pass receptors and are transported to the plasma membrane as cleaved. Notch intracellular domain (NICD) translocates to the nucleus, where it forms a complex with the DNA binding protein CSL, displacing a histone deacetylase (HDAc)-co-repressor (CoR) complex from CSL. Notch signaling pathway can either act oncogenic or in a tumor-suppressive manner.
APC (Wnt): Wnt proteins are secreted morphogens that are required for basic developmental processes, such as cell-fate specification, progenitor-cell proliferation and the control of asymmetric cell division, in many different species and organs.
RAS: The Ras proteins are GTPases that function as molecular switches for signaling pathways regulating cell proliferation, survival, growth, migration, differentiation or cytoskeletal dynamism.)
TGF-B: The transforming growth factor-beta (TGF-beta) family members, which include TGF-betas, activins and bone morphogenetic proteins (BMPs), are structurally related secreted cytokines. A wide spectrum of cellular functions such as proliferation, apoptosis, differentiation and migration are regulated by TGF-beta family members.
HedgeHog: The Hedgehog (Hh) family of secreted signaling proteins plays a crucial role in development, regulating morphogenesis of a variety of tissues and organs. Hh signaling is also involved in control of stem cell proliferation in adult tissues and aberrant activation of the Hh pathway has been linked to multiple types of human cancer. Members of the Hh family bind to patched (ptc), thus releasing smoothened (smo) to transduce a signal.
Transcriptional Regulation (KEGG): A collection of pathways known to be transcriptionally mis-regulated in a variety of cancers.
JAK/STAT: The Janus kinase/signal transducers and activators of transcription (JAK/STAT) pathway is a pleiotropic cascade used to transduce a multitude of signals for development and homeostasis in animals. It is the principal signaling mechanism for a wide array of cytokines and growth factors which leads to activation of additional transcription factors.
RAS: The Ras proteins are GTPases that function as molecular switches for signaling pathways regulating cell proliferation, survival, growth, migration, differentiation or cytoskeletal dynamism.)

The system and methods described herein may be applied to modulate the expression of one or more of the genes listed in Table 2. Microwave energy may be used to restore the expression of one or more genes which participate in immunomodulatory pathways.

### REFERENCES

[1] L. Coussens and Z. Werb, “Inflammation and cancer,” Nature., vol. 420, no. 6917, pp. 860-867, 2002.
[2] “SEER Training Modules, Module Name. U. S. National Institutes of Health, National Cancer Institute,” [Online]. Available: https://training.seer.cancer.gov. [Accessed 9 2 2021].
[3] G. Multhoff, M. Molls and J. Radons, “Chronic inflammation in cancer development,” Front Immunol., vol. 2, no. 98, 2012.
[4] R. Ness and C. Cottreau, “Possible Role of Ovarian Epithelial Inflammation in Ovarian Cancer,” Journal of the National Cancer Institute, vol. 91, no. 17, p. 1459-1467, 1999.
[5] X. Sui, L. Lei, L. Chen, T. Xie and X. Li, “Inflammatory microenvironment in the initiation and progression of bladder cancer,” Oncotarget., vol. 8, no. 54, pp. 93279-93294, 2017.
[6] M. Neagu, C. Constantin, C. Caruntu, C. Dumitru, M. Surcel and Z. S., “Inflammation: A key process in skin tumorigenesis,” Oncol Lett., vol. 17, no. 5, pp. 4068-4084, 2019.
[7] R. Okamoto, “Mini Review Epithelial regeneration in inflammatory bowel diseases,” Inflammation and Regeneration, vol. 31, no. 3, 2011.
[8] S. Savant, S. Sriramkumar and H. O'Hagan, “The Role of Inflammation and Inflammatory Mediators in the Development, Progression, Metastasis, and Chemoresistance of Epithelial Ovarian Cancer,” Cancers (Basel)., vol. 10, no. 8, p. 251, 2018.
[9] L. Murtaugh and M. Keefe, “Regeneration and repair of the exocrine pancreas,” Annu Rev Physiol., vol. 77, pp. 229-249, 2015.
[1 J. Liu, K. Madsen, P. Boulanger, L. Dieleman, J. Meddings and R. Fedorak, “Mind 0] The Gaps Confocal Endomicroscopy Showed Increased Density of Small Bowel Epithelial Gaps in Inflammatory Bowel Disease,” Journal of Clinical Gastroenterology, vol. 45, no. 3, p. 240-245, 2011.
[1 A. Sturm and A. Dignass, “Epithelial restitution and wound healing in inflammatory 1] bowel disease,” World J Gastroenterol, vol. 14, no. 3, pp. 348-353, 2008.
[1 T. Watanabe, M. Kudo and S. W, “Immunopathogenesis of pancreatitis,” Mucosal 2] Immunology, vol. 10, p. 283-298, 2017.
[1 M. Lizuka and S. Konno, “Wound healing of intestinal epithelial cells,” World Journal 3] of Gastroenterology, vol. 17, no. 17, pp. 2161-71, 2011.
[1 G. Pineton-de-Chambrun, L. Peyrin-Biroulet, M. Lemann and J. Colombel, “Clinical 4] implications of mucosal healing for the management of IBD,” Nat Rev Gastroenterol Hepatol., pp. 15-29, 2010.
[1 G. Lichtenstein and P. Rutgeerts, “Importance of mucosal healing in ulcerative 5] colitis,” Inflamm Bowel Dis., vol. 16, pp. 38-46, 2010.
[1 L. Peyrin-Biroulet, A. Bressenot and W. Kampman, “Histologic Remission: The 6] Ultimate Therapeutic Goal in Ulcerative Colitis?,” Clinical Gastroenterology and Hepatology, vol. 12, no. 6, pp. 929-34, 2014.
[1 K. Funk, M. Takeshi, T. Oshima, E. Park, M. Yagi and K. Tomizuka, “Method of 7] stimulating epithelial cell proliferation by administration of gastrointestinal proliferative factor”. U.S. Pat. No. 7,951,381B2, 31 May 2011.
[1 N. Toda, Y. Hidaka, M. Ishikawa and S. Kanno, “Preventive and/or ameliorative 8] agent for diseases, stamina enhancement agent, anti-fatigue agent, and pharmaceutical and food and drink using them”. U.S. Pat. No. 9,737,573B2, 22 Aug. 2017.
[1 M. Ferguson, H. Laverty, S. O′kane and N. Occleston, “Promotion of epithelial 9] regeneration”. Australia Patent AU2006268091C1, 18 Jul. 2007.
[2 P. Michieli, “Anti-MET antibody and its application”. USA Patent 0] US20190315873A1, 17 OCt. 2019.
[2 G. Ghatnekar, R. Gourdie and J. Jourdan, “Compositions and methods for 1] promoting wound healing and tissue regeneration”. U.S. Pat. No. 9,855,313B2, 2 Jan. 2018.
[2 M. Schwartz, “Treatment of intestinal epithelial cell malfunctions with Hepatocyte 2] Growth Factor”. U.S. Pat. No. 5,972,887A, 26 Oct. 1999.
[2 U. Martin, “Compositions and process for integrating cells into epithelium”. WIPO 3] Patent WO2020245277A1, 10 Dec. 2020.
[2 J.-G. Geng, W.-J. Zhou and L. Ma, “Compositions and methods relating to induction 4] of intestinal stem cell homeogenesis and/or regeneration”. U.S. Pat. No. 9,827,290B2, 28 Nov. 2017.
[2 C. Kuo, “Modulation of gastrointestinal epithelium proliferation through the Wnt 5] signaling pathway”. USA Patent US20050169995A1, 4 Aug. 2005.
[2 V. Mohanan, K. Lassen, M. Daly and R. Xavier, “Compositions and methods for 6] treating inflammatory bowel diseases”. WIPO Patent WO2019018410A1, 24 Jan. 2019.
[2 C. Hancock, “Microwave array applicator for hyperthermia”. Australia Patent 7] AU2007330615B2, 12 Jun. 2008.
[2 S. Flock and K. Marchitto, “Electromagnetic treatment of tissues and cells”. USA 8] U.S. Pat. No. 7,967,839B2, 28 Jun. 2011.
[2 A. Hezi-Yamit, S. Edwards and S. Sullivan, “Methods And Devices For Localized 9] Inhibition Of Inflammation By Ablation”. US Patent US 2015/0126978 A1, 7 May 2015.
[3 J. Caplan, H. Rajagopalan and J. Flaherty, “Electrical Energy Ablation Systems, 0] Devices And Methods For The Treatment Of Tissue”. US Patent US 2015/0141987 A1, 21 May 2015.
[3 P. Levin, J. Caplan, H. Rajagopalan, M. Manasas, A. Coats and J. Flaherty, “Heat 1] Ablation Systems, Devices And Methods For The Treatment Of Tissue”. U.S. Pat. No. 10,349,998 B2, 16 Jul. 2019.
[3 R. Sharp, D. Petersonm, D. Kerr, A. Ward, A. Ross, R. Coulson and W. Nau, 2] “Devices, Systems, And Methods For Treating Ulcerative Colitis And Other Inflammatory Bowel Diseases”. WIPO Patent WO 2017/087191 A1, 26 May 2017.
[3 R. Izutani, E. Loh, H. Reinecker, Y. Ohno, R. Fusunyan, L. GR, J. Rombeau and R. 3] Macdermott, “Increased expression of interleukin-8 mRNA in ulcerative colitis and Crohn's disease mucosa and epithelial cells,” Inflamm Bowel Dis., vol. 1, no. 1, pp. 37-47, 1995.
[3 R. Fernando, M. Castillo, M. Litzinger, D. Hamilton and C. Palena, “IL-8 signaling 4] plays a critical role in the epithelial-mesenchymal transition of human carcinoma cells,” Cancer Res., vol. 71, no. 15, pp. 5296-306, 2011.
[3 L. Y, C. de-Haar, M. Peppelenbosch and C. van-der-Woude, “SOCS3 in immune 5] regulation of inflammatory bowel disease and inflammatory bowel disease-related cancer,” Cytokine Growth Factor, vol. 23, no. 3, pp. 127-38, 2012.
[3 W. Yu, Z. Lin, J. Hegarty, X. Chen, A. Kelly, Y. Wang, L. Poritz and W. Koltun, 6] “Genes differentially regulated by NKX2-3 in B cells between ulcerative colitis and Crohn's disease patients and possible involvement of EGR1,” Inflammation, vol. 35, no. 3, pp. 889-99, 2012.
[3 F. Wu, T. Dassopoulos, L. Cope, A. Maitra, S. Brant, M. Harris, B. T. G. Parmigiani 7] and S. Chakravarti, “Genome-wide gene expression differences in Crohn's disease and ulcerative colitis from endoscopic pinch biopsies: insights into distinctive pathogenesis,” Inflamm Bowel Dis., vol. 13, no. 7, pp. 807-21, 2007.
[3 C. Andrews, M. Mclean and S. Durum, “Cytokine Tuning of Intestinal Epithelial 8] Function,” Front Immunol., vol. 9, p. 1270, 2018.
[3 R. Li, S. Ong and L. Tran, “Chronic IL-1B-induced inflammation regulates epithelial-9] to-mesenchymal transition memory phenotypes via epigenetic modifications in non-small cell lung cancer,” Sci Rep, vol. 10, p. 377, 2020.
[4 C. Braegger, S. Nicholls, S. Murch, S. Stephens and T. MacDonald, “Tumour 0] necrosis factor alpha in stool as a marker of intestinal inflammation,” Lancet, vol. 339, no. pmid: 1345871, pp. 89-91, 1992.
[4 H. Masuda, S. Iwai, T. Tanaka and S. Hayakawa, “Expression of IL-8, TNF-alpha 1] and IFN-gamma m-RNA in ulcerative colitis, particularly in patients with inactive phase,” J Clin Lab Immunol., vol. 46, no. pmid:8926619, p. 111-123, 1995.
[4 F. Balkwill, “TNF-α in promotion and progression of cancer,” Cancer Metastasis Rev 2] 25, vol. 409, 2006.
[4 J. Ryan, W. Linde-Zwirble, L. Engelhart, L. Cooper and D. Cohen, “Temporal 3] changes in coronary revascularization procedures, outcomes, and costs in the bare-metal stent and drug-eluting stent eras: results from the us medicare program,” Circulation, p. 119:952-9, 2009.
[4 G. Tepe, U. Beschorner, C. Ruether, I. Fischer, P. Pfaffinger, E. Noory and T. Zeller, 4] “T. Drug-eluting balloon therapy for femoropopliteal occlusive disease: predictors of outcome with a special emphasis on calcium,” J Endovasc Ther, vol. 22, p. 727-733, 2012.
[4 U. Bildirici, M. Aktas, E. Dervis and U. çelikyurt, “Mid-Term Outcomes of Stent 5] Overlap in Long Total Occluded Lesions of Superficial Femoral Artery,” Med Sci Monit, vol. 23, pp. 3130-3135, 2017.
[4 B. Alyavi and J. Uzokov, “Peripheral artery disease in the lower extremities: 6] indications for treatment,” E-Journal-of-Cardiology-Practice, vol. 16, 2018.
[4 P. R. Kornaat, R. Sharma, R. J. v. d. Geest, H. J. Lamb, M. Kloppenburg, M.-P. H. 7] I. Graverand, J. L. Bloem and I. Watt, “Positive association between increased popliteal artery vessel wall thickness and generalized osteoarthritis: is OA also part of the metabolic syndrome?,” Skeletal Radiology, vol. 38, pp. 1147-58, 2009.
[4 Y. G. Wolf, Z. Kobzantsev and L. Zelmanovich, “Size of normal and aneurysmal 8] popliteal arteries: A duplex ultrasound study,” JOURNAL OF VASCULAR SURGERY, vol. 43, no. 3, pp. 488-492, 2006.
[4 R. Debasso, H. Åstrand, N. Bjarnegård, Å. R. Ahlgren and T. Sandgren, “The 9] popliteal artery, an unusual muscular artery with wall properties similar to the aorta: Implications for susceptibility to aneurysm formation?,” JOURNAL OF VASCULAR SURGERY, vol. 39, no. 4, pp. 836-842, 2003.
[5 A. V. Telang, M. L. and M. Natarajan, “A study of the internal diameter of popliteal 0] artery, anterior and posterior tibial arteries in cadavers,” Indian Journal of Clinical Anatomy and Physiology, vol. 3, no. 3, pp. 287-290, 2016.
[5 “Leaky” Cables Make Fine Broadband Antennas, microwavejournal,” May 3, 2015. 1] [Online]. Available: https://www.gore.com/sites/g/files/ypyipe116/files/2016-05/Leaky %20Cables %20Make %20Fine %20Broadband %20Antennas %20-%2020 15-03-05%20-%20Microwave %20Journal.pdf. [Accessed Jan. 5, 2019].
[5 S. MAINI and A. MARWAHA, “Development and Analysis of Coaxial Slot Antenna 2] in Comparison with Coaxial Dipole Antenna for Interstitial Microwave Ablation,” in WORLD-EDU′12/CIT′WORLD-EDU′12/CIT′12 Proceedings of the 6th international conference on Communications and Information Technology, Athens, Greece, 2012.
[5 M. F. J. C. Rubio, A. V. Hernández, L. L. Salas, E. Ávila-Navarro and E. Navarro, 3] “Coaxial Slot Antenna Design for Microwave Hyperthermia using Finite-Difference Time-Domain and Finite Element Method,” The Open Nanomedicine Journal, vol. 3, pp. 2-9, 2011.
[5 M. Sawarbandhe, B. Naik, V. Satpute and S. S, “Coaxial Antenna for Microwave 4] Ablation,” in IEEE Distributed Computing, VLSI, Electrical Circuits and Robotics (DISCOVER), Mangalore, India, 2016.
[5 O. Mogabgab, V. Patel, T. Michael and K. A, “Impact of Contrast Agent Viscosity on 5] Coronary Balloon Deflation Times: Bench Testing Results,” J Interv Cardiol, vol. 27, no. 2, pp. 177-181, 2014.
[5 G. Leibundgut, C. Degen and R. F, “Transcutaneous Puncture of an Undeflatable 6] Coronary Angioplasty Balloon Catheter,” Case Reports in Cardiology, vol. Article ID 6252809, p. 5, 2018.
[5 “Contrast Media,” General Electric, July 2018. [Online]. Available: 7] https://www.gehealthcare.co.uk/sitecore/content/gehc/home/products/contrast-media?sc_lang=en. [Accessed 29 5 2019].
[5 F. Suzuki, K. Takano, N. Ishii, J. Kumada, Y. Ozaki, Y. Nasu and K. Satou, “Leaky 8] Coaxial Cable”. U.S. Pat. No. 8,378,764 B2, 2013.
[5 F. Rossetto, J. Brannan, J. Paulus and C. Debroski, “Leaky-wave Antennas For 9] Medical Applications”. U.S. Pat. No. 8,202,270 B2, 2012.
[6 J. Krueger, P. Ewert, S. Yilmaz, D. Gelernter, B. Peters, K. Pietzner, A. Bornstedt, 0] B. Schnackenburg, H. Abdul-Khaliq, E. Fleck, E. Nagel, B. F and T. Kuehne, “Magnetic Resonance Imaging-Guided Balloon Angioplasty of Coarctation of the Aorta,” Circulation, vol. 113, no. 8, pp. 1093-1100, 2006.
[6 D. Yin, M. Matsumura, j. Rundback, J. Yoho, B. Witzenbichler, G. Stone, G. Mintz 1] and A. Maehara, “Comparison of plaque morphology between peripheral and coronary artery disease (from the CLARITY and ADAPT-DES IVUS substudies),” Coron Artery Dis., vol. 28, no. 5, pp. 369-375, 2017.
[6 Gurm, C. Kasapis and S. Hitinder, “Current Approach to the Diagnosis and 2] Treatment of Femoral-Popliteal Arterial Disease. A Systematic Review,” Curr Cardiol Rev., vol. 5, no. 4, p. 296-311, 2009.
[6 A. E. Simone Biscaglia, D. Bernucci, G. Bugani, E. Favaretto and G. Campo, “BRS 3] implantation in long lesions requiring device overlapping: myth or reality?,” Journal of Thoracic Disease (Bioresorbable Vascular Scaffolds for the Treatment of Coronary Artery Disease)/, vol. 9 supp 9, 2017.
[6 P. Gas, “Optimization of multi-slot coaxial antennas for microwave thermotherapy 4] based on the S11-parameter analysis,” Biocybernetics and Biomedical Engineering, vol. 37, no. 1, pp. 78-93, 2017.
[6 Y. Jiang, J. Zhao, W. Li, Y. Yang, J. Liu and Z. Qian, “A coaxial slot antenna with 5] frequency of 433 MHz for microwave ablation therapies: design, simulation, and experimental research,” Med Biol Eng Comput, vol. 55, no. 11, p. 2027-2036, 2017.
[6 C. Heng-mao, S. Ananda Mohan, T. Phillip, R. Leslie and G. Ramsay, “A Microwave 6] Antenna For Medical Ablation”. European Patent Office Patent EP 1613230 B1, 2010.
[6 G. Karczewski, M. Kopcewicz and A. Kotlicki, “Skin depth effect in the RF collapse 7] studies,” J. Phys. Colloques, vol. 41, pp. 217-218, 1980.
[6 “OMNIPAQUE™ (iohexol) Injection 140 180 240 300 350, FDA: Reference ID: 8] 4080358,” General Electric, 2017. [Online]. Available: https://www.accessdata.fda.gov/drugsatfda_docs/label/2017/018956s099lbl.pdf. [Accessed 29 5 2019].
[6 K. Katsanos, S. Spiliopoulos, P. Kitrou, M. Krokidis and D. Karnabatidis, “Risk of 9] Death Following Application of Paclitaxel-Coated Balloons and Stents in the Femoropopliteal Artery of the Leg: A Systematic Review and Meta-Analysis of Randomized Controlled Trials,” vol. 7, no. 24, p. e011245, 2018.
[7 V. Ng, C. Mena, C. Pietras and L. AJ, “Local delivery of paclitaxel in the treatment 0] of peripheral arterial disease,” Eur J Clin Invest., vol. 45, no. 5, pp. 333-45, 2015.
[7 J. J. Dodge, B. Brown, E. Bolson and H. Dodge, “Lumen diameter of normal human 1] coronary arteries. Influence of age, sex, anatomic variation, and left ventricular hypertrophy or dilation,” Circulation, vol. 86, no. 1, pp. 232-46, 1992.
[7 J. Q. Del-Rosso, “Topical Imiquimod Therapy for Actinic Keratosis. Is Long-Term 2] Clearance a Realistic Benefit?,” J Clin Aesthet Dermatol., vol. 1, no. 3, pp. 44-47, 2008.
[7 S. Adams, L. Kozhaya, F. Martiniuk, T. Meng, L. Chiriboga, L. Liebes, T. Hochman, 3] N. Shuman, D. Axelrod, J. Speyer, Y. Novik, A. Tiersten, J. Goldberg, C. Formenti, N. Bhardwaj, D. Unutmaz and S. Demaria, “Topical TLR7 agonist imiquimod can induce immune-mediated rejection of skin metastases in patients with breast cancer,” Clin Cancer Res., vol. 18(24), no. 24, pp. 6748-57, 2012.
[7 A. H. GmbH, “DICLOFENAC SODIUM 3% GEL,” 2019. [Online]. Available: 4] https://www.drugs.com/uk/diclofenac-sodium-3-gel-leaflet.html. [Accessed 17 Oct. 2019].
[7 K. Thai, P. Fergin, M. Freeman, C. Vinciullo, D. Francis, L. Spelman, D. Murrell, C. 5] Anderson, W. Weightman, C. Reid, A. Watson and P. Foley, “A prospective study of the use of cryosurgery for the treatment of actinic keratoses,” Int J Dermatol., vol. 43(9), no. 9, pp. 687-92., 2004 September
[7 A. Dodds, A. Chia and S. Shumack, “Actinic Keratosis: Rationale and Management,” 6] Dermatol Ther (Heidelb), pp. 11-31, 2014.
[7 N. Krawtchenko, J. Roewert-Huber, M. Ulrich, I. Mann, W. Sterry and E. Stockfleth, 7] “A randomised study of topical 5% imiquimod vs. topical 5-fluorouracil vs. cryosurgery in immunocompetent patients with actinic keratoses: a comparison of clinical and histological outcomes including 1-year follow-up,” Br J Dermatol., vol. 157, pp. Suppl 2:34-40, 2007.
[7 “NanoString Technologies,” Seattle, Washington, United States, 2019. [Online]. 8] Available: https://www.nanostring.com. [Accessed 17 Oct. 2019].
[7 “ClinicalTrials.gov, Identifier: NCT03483935,” U.S. National Library of Medicine, 30 9] 3 2018. [Online]. Available: https://clinicaltrials.gov/ct2/show/NCT03483935?term=microwave&cond=Actinic+K eratoses&rank=1. [Accessed 17 Oct. 2019].
[8 X. L. Wang, H. W. Wang, K. Yuan, F. L. Li and Z. Huang, “Combination of 0] photodynamic therapy and immunomodulation for skin diseases-update of clinical aspects,” Photochemical & Photobiological Sciences, vol. 10, no. 5, p. 704, 2011.
[8 U. R. Hengge and T. Ruzicka, “Topical Immunomodulation in Dermatology: 1] Potential of Toll-like Receptor Agonists,” Dermatologic Surgery, vol. 30, no. 8, pp. 1101-1112, 2004.
[8 J. W. Hadden, “Immunopharmacology,” JAMA, vol. 268, no. 20, pp. 2964-69, 1992. 2]
[8 S. Reitamo, A. Remitz, H. Kyllönen and J. Saarikko, “Topical noncorticosteroid 3] immunomodulation in the treatment of atopic dermatitis,” Am J Clin Dermatol., vol. 3, no. 6, pp. 381-8, 2002.
[8 S. H. Ibbotson, “Topical 5-aminolaevulinic acid photodynamic therapy for the 4] treatment of skin conditions other than non-melanoma skin cancer,” British Journal of Dermatology, vol. 146, no. 2, pp. 178-188, 2002.
[8 X. Li, X. Gao, L. Jin, Y. Wang, U. Hong, X. McHepange, Y.. Wang, U. Jiang, J. Wei 5] and H.-D. Chen, “Local hyperthermia could induce migrational maturation of Langerhans cells in condyloma acuminatum,” J Dermatol Sci, vol. 54, no. 2, pp. 121-3, 2009.
[8 S. Corr, N. Jared, C. Steven and A. Sikora, “Radiofrequency Field Hyperthermia 6] And Solid Tumor Immunomodulation”. WIPO Patent WO 2018/071837 A1, 19 Apr. 2018.
[8 J. Tyler, A. Jaeger, S. Santagata, L. Whitesell and S. Lindquist, “Combination 7] Treatments Of Hsp90 Inhibitors For Enhancing Tumor Immunogenicity And Methods Of Use Thereof”. WIPO Patent WO 2019/232533 A1, 5 Dec. 2019.
[8 S. Ferree, J. Cowens, C. Jorgensen, T. Nielsen and B. Ejlertsen, “Methods Of 8] Treating Breast Cancer With Gemcitabine Therapy”. US Patent US 2014/0037620 A1, 6 Feb. 2014.
[8 L. Radvanyi, J. Chen and H. Patrick, “Biomarkers And Targets For Cancer 9] Immunotherapy”. WIPO Patent WO 2016/073748 A1, 12 May 2016.
[9 F.-C. Tsai, G.-J. Chang, Y.-J. Hsu, Y.-M. Lin, Y.-S. Lee, W.-J. Chen, C.-T. Kuo and 0] Y.-H. Yeh, “Proinflammatory gene expression in patients undergoing mitral valve surgery and maze ablation for atrial fibrillation,” The Journal of Thoracic and Cardiovascular Surgery, vol. 151, no. 6, pp. 1673-1682.e5, 2015.
[9 J. Feehan, S. Burrows, L. Cornelius, A. M. Cook, K. Mikkelsen, V. Apostolopoulos, 1] M. Husaric and D. Kiatos, “Therapeutic applications of polarized light: Tissue healing and immunomodulatory effects,” Maturitas, vol. 116, pp. 11-17, 2018.
[9 Q. Huang, J. Hu, F. Lohr, L. Zhang, R. Braun, J. Lanzen, J. Little, M. Dewhirst and 2] C. Li, “Heat-induced gene expression as a novel targeted cancer gene therapy strategy,” Cancer Res, vol. 60, no. 13, pp. 3435-9, 2000.
[9 F. Barrat, “Methods Of Treating Cancer, Infectious Disease, And Autoimmune 3] Disease Using Cxc Chemokines”. US Patent US 2018/0193382 A1, 12 Jul. 2018.

1. Use of microwave energy or a microwave system or microwave-generating apparatus, for modulating the expression of one or more genes.

2. The use of claim 1, wherein the microwave system or microwave generating apparatus comprises:
a microwave generator;
a controller configured to control the microwave generator to generate microwave energy having a selected operational frequency or range of frequencies;
a microwave energy conduit cable configured to deliver the microwave energy to a microwave antenna extending from or coupled to a distal end of the microwave energy conduit cable; and
a microwave antenna.

3. The use of claim 1 or 2, wherein the microwave system or microwave generating apparatus is used to deliver or administer microwave energy to a tissue, to a biopsy or to a diseased tissue.

4. The use of claim 3, wherein the diseased tissue, biopsy or diseases tissue, comprises diseased epithelial tissue.

5. The use of any claim 1, wherein the microwave energy is used at a frequency of:
between about 300 MHz and about 300 GHz; or
between about 900 MHz and about 200 GHz; or
between about 900 MHz and about 15 GHz; or
about 14 GHz; or
about 13 GHz; or
about 12 GHz; or
about 11 GHz; or
about 10 GHz; or
about 9 GHz; or
about 7.5 GHz to about 8.5 GHz; or
about 8.5 GHz; or
about 8 GHz; or
about 7.5 GHz; or
about 7 GHz; or
about 6 GHz; or
about 5.8 GHz; or
about 2.45 GHz.

6. The use claim 5, wherein the microwave energy is used for about 1-5s, or for about 2-8s, or for about 3-10s, for about 1s, for about 2s, for about 5 s, for about 10 s, for about 30 s, for about 60 s, for about 2 min, for about 5 min or for about 10 min.

7. The use of claims 5-6, wherein the microwave energy is used at a very low energy level energy, or at 10-50 J, or at 30-80 J or at 50 J-100 J.

8. The use of claims 5-7, wherein the microwave energy is used at 0.1 W to 50 W, about 1 W, about 2 W, about 5 W, about 8 W, about 10 W, about 15 W, about 20 W, about 25 W, about 30 W or about 40 W.

9. The use of claims 5-8, wherein the microwave energy is used at a plurality of different powers and/or wherein the microwave energy is used at different powers and adjusted between different powers in increments of 0.1 W.

10. The use of any one of claims 1-9, wherein the microwave energy is used as a repeat dose.

11. The use of claim 10, wherein the microwave energy is used as 2, 5, 10 or 15 individual doses of microwave energy.

12. The use of claim 11, wherein the duration of each dose may be the same or different.

13. The use of claim 11 or 12, wherein there is a pause between each dose.

14. The use of claim 13, wherein the duration of the pause between each dose may be the same or different

15. The use of claim 13 or 14, wherein the duration of the pause can be anywhere between about 1 s, 5 s, 10 s, 20 s, 60 s, typically between 5 s-20 s.

16. The use of any one of claims 5-15, wherein the microwave energy is applied to:
a tissue; or
a tissue sample; or
a tissue provided or obtained by/from a subject; or
a biopsy.

17. The use of claim 16, wherein the tissue is selected from the group consisting of:
(a) epithelial tissue;
(b) Simple squamous epithelium;
(c) Simple cuboidal epithelium;
(d) Simple columnar epithelium;
(e) Pseudostratified epithelium;
(f) Stratified squamous epithelium;
(g) Non-keratinized;
(h) Keratinized;
(i) Stratified cuboidal epithelium;
(j) Stratified columnar epithelium;
(k) Transitional epithelium; and
(l) Germinal simple squamous-to-cuboidal epithelium.

18. The use of claim 16 or 17, wherein the microwave energy is used to deliver or administer a thermal effect to the tissue.

19. The use of any preceding claim, wherein the use is an in vitro use and/or the microwave energy is applied or administered to a tissue or biopsy in vitro.

20. The use of claim 18, wherein the microwave energy is used to raise the temperature in the tissue from a first temperature to a second temperature.

21. The use of claim 20, wherein the first temperature may be equal to the temperature of the untreated tissue and the second temperature is higher than the first temperature.

22. The use of claim 20 or 21, wherein the first temperature is about 35° C., 36° C., 37° C. 38° C., 39° C. or about 40° C.; and the second temperature is be about 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C. or 59° C., 60° C. or about 61ºC.

23. The use of any one of claims 20-22, wherein the second temperature may not reach 60° C.

24. The use of any one of claims 20-23, wherein the microwave energy is be used to raise the temperature in a tissue from about 37° ° C. to about 59° C.

25. The use of any one of claims 20-24, wherein the second temperature is maintained for a period of time, for anywhere between about 1s and 30 min, for about 1 min, for about 5 min, for about 10 min, for about 15 min, for about 20 min or for about 25 min.

26. The use of any one of claims 20-25, wherein temperature of the tissue is raised from a first temperature equal to the temperature of the tissue before application of any microwave based treatment to a second temperature of about 43° C.

27. The use of any one of claims 20-26, wherein the microwave energy is used as a series of alternate high and low microwave power pulses.

28. The use of any one of claims 20-27, wherein the microwave energy is used at 20 W for 10 s to raise the temperature of the tissue from a first temperature to a second temperature.

29. The use of claim 28, wherein the second temperature is 43° C.

30. The use of claim 28 or 29, wherein the second temperature is maintained using microwave energy at 2 W.

31. A method of modulating gene expression in a subject or in a tissue, said method comprising exposing the subject or tissue to microwave energy.

32. The method of claim 31, wherein the subject is a human or animal subject.

33. The method of claim 31 or 32, wherein the subject is suffering from or susceptible/predisposed to an inflammatory disease or condition.

34. The method of any one of claim 31 or 32, wherein the subject is suffering from or susceptible/predisposed to, a disease or condition which is caused by and/or associated with, the aberrant expression of one or more of:
the IL8 gene; and/or
the SOCS3 gene; and/or
the EGR1 gene; and/or
the CD79A gene; and/or
the IL1B gene; and/or
the TNFRSF13C gene.

35. The method of any one of claims 31-34 wherein the microwave energy is used or administered at a dose sufficient to modulate the expression of the relevant gene.

36. The method of any one of claims 31-35, wherein the method is used to modulate the expression of one or more genes in a tissue.

37. The method of any one of claims 31-36, wherein the method is used to restore the expression of a gene.

38. A method of treating or preventing a disease or condition caused by and/or associated with:
aberrant expression of the IL8 gene
aberrant expression of the SOCS3 gene
aberrant expression of the EGR1 gene
aberrant expression of the CD79A gene
aberrant expression of the IL1B gene
aberrant expression of the TNFRSF13C gene;
said method comprising administering a subject in need of treatment, microwave energy.

39. The method of claim 38, wherein the microwave energy is administered at a dose sufficient to modulate the expression of the relevant gene.

40. A method of upregulating the expression of one or more of the following gene(s):
the KIT gene; and/or
the BAD gene; and/or
the ID4 gene; and/or
the RUNX1T1 gene; and/or
the AKT3 gene;
said method comprising exposing the relevant gene to microwave energy.

41. A method or in vitro method of down-regulating the expression of one or more of the following gene(s):
the IL8 gene; and/or
the SOCS 3 gene; and/or
the EGR1 gene; and/or
the CD79A gene; and/or
the IL1B gene; and/or
the TNFRSF13C gene; and/or
the GADD45B gene; and/or
the Notch3 gene; and/or
the CCND2 gene; and/or
the WNT5A gene;
said method comprising exposing the relevant gene to microwave energy.

42. The methods of claim 40 or 41, wherein the method(s) is/are applied to a tissue or to an isolated tissue sample.

43. A method or in vitro method of treating or preventing a disease or condition caused by and/or associated with:
downregulated expression of the KIT gene; and/or
downregulated expression of the BAD gene; and/or
downregulated expression of the ID4 gene; and/or
downregulated expression of the RUNX1T1 gene; and/or
downregulated expression of the AKT3 gene;
said method comprising administering a subject suffering from or predisposed/susceptible to any such disease, microwave energy.

44. The method of claim 43, wherein the microwave energy is administered at a dose sufficient to upregulate and/or restore the expression of the relevant gene, wherein the expression of the gene is upregulated and/or restored to the extent that the upregulated/restored expression matches the expression of the same gene in a normal or healthy subject/tissue.

45. A method of treating or preventing a disease or condition caused by and/or associated with:
upregulated expression of the IL8 gene; and/or
upregulated expression of the SOCS3 gene; and/or
upregulated expression of the EGR1 gene; and/or
upregulated expression of the CD79A gene; and/or
upregulated expression of the IL1B gene; and/or
upregulated expression of the TNFRSF13C gene; and/or
upregulated expression of the GADD45B gene; and/or
upregulated expression of the Notch3 gene; and/or
upregulated expression of the CCND2 gene; and/or
upregulated expression of the WNT5A gene;
said method comprising administering a subject suffering from or predisposed/susceptible to any such disease, microwave energy.

46. The method of claim 45, wherein the microwave energy is administered at a dose sufficient to downregulate and/or restore the expression of the relevant gene, wherein the expression of the gene is downregulated and/or restored to the extent that the downregulated/restored expression matches the expression of the same gene in a normal or healthy tissue.

47. An in vitro method of modulating gene expression, said method comprising exposing the gene to microwave energy.

48. A method of modulating:
the PI3K pathway; and/or
the RAS pathway; and/or
the MAPK pathway; and/or
the Notch pathway; and/or
the Wnt pathway; and/or
the Transcriptional Regulation (KEGG) pathway; and/or
the JAK/STAT pathway; and/or
the TGF-B pathway; and/or
the Hedgehog pathway;
said method comprising exposing the pathway to microwave energy at a dose sufficient to modulate the expression of the relevant pathway.

49. A method of treating or preventing a disease or condition caused by and/or associated with:
aberrant expression of the PI3K pathway; and/or
aberrant expression of the RAS pathway; and/or
aberrant expression of the MAPK pathway; and/or
aberrant expression of the Notch pathway; and/or
aberrant expression of the Wnt pathway; and/or
aberrant expression of the Transcriptional Regulation (KEGG) pathway; and/or
aberrant expression of the JAK/STAT pathway; and/or
aberrant expression of the TGF-B pathway; and/or
aberrant expression of the Hedgehog pathway; and/or
said method comprising administering a subject in need of treatment, microwave energy at a dose sufficient to modulate the expression of the relevant pathway.

50. Use of microwave energy to upregulate the expression of one or more of the following gene(s):
the KIT gene; and/or
the BAD gene; and/or
the ID4 gene; and/or
the RUNX1T1 gene; and/or
the AKT3 gene.

51. Use of microwave energy to down-regulate the expression of one or more of the following gene(s):
the IL8 gene; and/or
the SOCS 3 gene; and/or
the EGR1 gene; and/or
the CD79A gene; and/or
the IL1B gene; and/or
the TNFRSF13C gene; and/or
the GADD45B gene; and/or
the Notch3 gene; and/or
the CCND2 gene; and/or
the WNT5A gene;
said method comprising exposing the relevant gene to microwave energy.

52. The use of claim 50 or 51, wherein the microwave energy is applied to a tissue, an isolated tissue, a biopsy or a tissue sample to up- or down-regulate the relevant gene expression as required.

53. The use of any one of claims 50-52, wherein the microwave energy is applied or administered at a frequency, duration, power or dose defined in any one of claims 5-15 and 18-30.
